(19) United States
(12) Patent Application Publication
(10) Pub. No.: US 2011/0091587 A1
(43) Pub. Date: Apr. 21, 2011

(54) PHASE II DETOXIFICATION AND ANTIOXIDANT ACTIVITY

(75) Inventors: T. Keith Blackwell, Waban, MA (US); Motonobu Matsumoto, Brookline, MA (US); Taketoshi Makino, Osaka (JP); Masashi Goto, Osaka (JP); Atsushi Ishikado, Osaka (JP); Mariko Maeda, Osaka (JP); Satoe Azechi, Osaka (JP)

(73) Assignees: JOSLIN DIABETES CENTER, INC., Boston, MA (US); SUNSTAR INC., Osaka (JP)

(21) Appl. No.: 12/677,045

(22) PCT Filed: Sep. 11, 2008
(86) PCT No.: PCT/US08/76064

§ 371 Date: Nov. 22, 2010

Related U.S. Application Data
(60) Provisional application No. 60/993,325, filed on Sep. 11, 2007.

(51) Int. Cl. A61K 36/76; C12Q 1/00; C12Q 1/68; A61P 39/06

(52) U.S. Cl. 424/771; 435/4; 435/6

(57) ABSTRACT

Provided are methods and compositions that enhance Nrf2 (SKN-1) activation of phase II detoxification or antioxidant enzyme transcription, comprising plant extracts (e.g., willow extracts) or active fractions thereof, as well as methods for identifying additional compounds that increase the Nrf2-regulation of those enzymes.

### CLAIM OF PRIORITY

This application claims the benefit of U.S. Provisional Patent Application Ser. No. 60/993,325, filed on Sep. 11, 2007, the entire contents of which are hereby incorporated by reference.

### TECHNICAL FIELD

The present invention relates to an antioxidant and detoxification function-enhancing action of willow, tea, and extracts thereof.

### BACKGROUND

Living bodies are constantly being exposed to various substances that can cause ill effects. Such substances include, for example, heavy metals, certain food additives, ultraviolet rays, and tobacco. When these substances act on the living body, reactive oxygen species known as free radicals are produced. The living body is further exposed to the oxidative stress it produces itself as a byproduct of certain physiological processes. Oxidative stress is considered as one of the risk factors for a number of conditions such as cancers, common diseases, and symptoms of aging. The living body deals with such oxidative stresses using a mechanism by which the free radicals are scavenged and toxic substances are detoxified (referred to herein as a host defense mechanism). When this mediation/detoxification mechanism is impaired, e.g., as a result of normal aging processes, the defense mechanism fails to completely mediate and detoxify these chemicals, a process which can sometimes lead to the onset of disease.

To solve this problem, methods for preventing development or progression of disease have been attempted that include taking or applying a substance having an antioxidant effect (e.g., compositions including vitamins C and/or E). These methods are valid; however, ingestion of large amounts of antioxidant substances are often required in order to produce clinically significant effects.

On the other hand, once enhanced, the host defense mechanism mentioned above can efficiently remove the oxidative stress, and is hence expected to be more useful than taking antioxidant substances. “Nrf2”, an intranuclear transcription factor, has attracted much interest as a critical protein that regulates the host defense mechanism. When a cell is exposed to oxidative stress or toxic substances, Nrf2 molecules present in the cytoplasm of the cell are imported into the nucleus, where they bind to a gene regulatory region known as an antioxidant response element (see, e.g., Nguyen, et al., Ann. Rev. Pharm. Toxicol., 2003, 43:233-60), and induce the expression of oxidative stress response enzymes, known as the phase II detoxification enzymes, which are present downstream of a sequence known as the antioxidant response element. Animals lacking the Nrf2 gene are known to have an impaired host defense mechanism. Nrf2 thus plays a critical role in the host defense mechanism against oxidative stress and toxic substances.

### SUMMARY

The present inventors have found that substances in certain plant extracts activate SKN-1/Nrf2 and strongly induce expression of Phase II detoxification enzyme (P2D) genes, decrease levels of 8-hydroxy-2′-deoxyguanosine (8-OHdG), and increase levels of forkhead box O1 (FOXO1) gene expression.

Thus, in one aspect the invention features methods and compositions for enhancing the activity of the P2D and antioxidant enzymes, e.g., compositions including plant extracts, e.g., extracts of willow, green tea, carrot, or broccoli, and/or active fractions thereof. In another aspect, the invention features methods of identifying substances that activate SKN-1/Nrf2, and therefore enhance P2D gene expression. As used herein, an “active fraction” is a fraction of the extract that has increased activity per weight as compared to the non-fractionated extract.

In one aspect, the invention provides compositions including plant extracts, e.g., extracts of willow, green tea, carrot, or broccoli, or an active fraction thereof, wherein the composition increases expression of one or both of a phase II detoxification enzyme (P2D) gene and an antioxidant enzyme gene in a cell. For example, the composition can increase expression of a P2D gene selected from the group consisting of glutamate-cysteine ligase modifier subunit (GCLM), and glutamate-cysteine ligase catalytic subunit (GCLC); and/or an antioxidant gene, e.g., superoxide dismutase 1 (SOD1). In some embodiments, the composition also increases expression of FOXO1, decreases levels of 8-hydroxy-2′-deoxyguanosine (8-OHdG), or both.

In some embodiments, the composition is formulated for oral administration, and can also include one or more orally acceptable carriers and additives. In some embodiments, the composition is formulated for topical administration, and can also include one or more topically acceptable carriers and additives.

In a further aspect, the invention provides methods for increasing the phase II detoxification enzyme (P2D) and/or antioxidant gene enhancing activity of an extract of willow. The methods include providing an extract of willow having a first level of P2D enhancing activity; fractionating the extract, to obtain two or more fractions; selecting a fraction having an Rf value of 0.5 or greater; assaying the P2D enhancing activity of the fraction; and selecting the fraction if it has a level of P2D enhancing activity that is higher than the first level of P2D enhancing activity.

In some embodiments, fractionating the extract comprises using one or more methods selected from the group consisting of column chromatography, liquid-liquid fractionation, and solid-liquid fractionation.

In yet another aspect, the invention provides methods of identifying a compound that increases expression of phase II detoxification enzyme (P2D) or antioxidant genes in a cell. The methods include providing a cell expressing (i) a P2D or antioxidant gene or (ii) a reporter construct comprising a P2D or antioxidant gene promoter, e.g., a Nrf2 binding sequence of a P2D gene promoter; providing a fraction of a plant extract; contacting said cell with said fraction; and detecting an effect of said fraction on expression of the P2D or antioxidant gene or reporter construct. A fraction that increases expression of the P2D or antioxidant gene or reporter construct comprises a compound that increases expression of phase II detoxification enzyme (P2D) and/or antioxidant genes in a cell.

In some embodiments, the methods also include selecting a fraction that increases expression of the P2D or antioxidant gene or reporter construct, and further dividing said fraction, to produce two or more subfractions; providing a cell expressing a P2D or antioxidant gene or a reporter construct comprising a P2D or antioxidant gene promoter, e.g., a Nrf2 binding sequence of a P2D gene promoter; contacting said cell with said subfraction; and detecting an effect of said subfraction on expression of the P2D or antioxidant gene or reporter construct. A subfraction that increases expression of the P2D or antioxidant gene or reporter construct comprises a compound that increases expression of phase II detoxification enzyme (P2D) and/or antioxidant genes in a cell. These steps can optionally be repeated until a purified compound is obtained, or a purified compound can be identified and obtained using standard split-pool methods.

In some embodiments, the methods also include formulating said purified compound for oral or topical administration.

In some embodiments, the cells used in these methods are cultured cells, peripheral blood mononuclear cells (PBMC), or cells in a Caenorhabditis elegans (e.g., an ASI cell).

In some embodiments, the plant extract is a willow extract. In some embodiments, the P2D gene is selected from the group consisting of glutamate-cysteine ligase modifier subunit (GCLM), glutamate-cysteine ligase catalytic subunit (GCLC). These methods can also be performed using an antioxidant gene, e.g., superoxide dismutase 1 (SOD1).

In some embodiments, the methods also include selecting a fraction that increases expression of the P2D or antioxidant gene or reporter construct, and further dividing said fraction, to produce two or more subfractions; providing a cell expressing a FOXO1 gene or a reporter construct comprising a FOXO1 gene promoter; contacting said cell with said subfraction; detecting an effect of said subfraction on expression of the FOXO1 gene or reporter construct; and selecting a subfraction that increases expression of the FOXO1 gene or reporter construct.

In some embodiments, the methods also include selecting a fraction that increases expression of the P2D or antioxidant gene or reporter construct, and further dividing said fraction, to produce two or more subfractions; contacting a cell with said subfraction; detecting an effect of said subfraction on levels of 8-hydroxy-2′-deoxyguanosine (8-OHdG) in the cell; and selecting a subfraction that reduces levels of 8-OHdG in the cell.

In an additional aspect, the invention provides methods of identifying a compound that increases expression of a forkhead box O1 (FOXO1) gene in a cell. The methods include providing a cell expressing (i) a FOXO1 gene or (ii) a reporter construct comprising a FOXO1 gene promoter; providing a fraction of a plant extract; contacting said cell with said fraction; and detecting an effect of said fraction on expression of the FOXO1 gene or reporter construct. A fraction that increases expression of the FOXO1 gene or reporter construct comprises a compound that increases expression of FOXO1 in a cell.

In some embodiments, the methods further include selecting a fraction that increases expression of the FOXO1 gene or reporter construct, and further dividing said fraction, to produce two or more subfractions; providing a cell expressing a FOXO1 gene or a reporter construct comprising a FOXO1 gene promoter; contacting said cell with said subfraction; and detecting an effect of said subfraction on expression of the FOXO1 gene or reporter construct. A subfraction that increases expression of the FOXO1 gene or reporter construct comprises a compound that increases expression of FOXO1 in a cell. These steps can be repeated until a purified compound is obtained, or other methods can be used for identifying a purified active compound, e.g., split-pool methods.

In some embodiments, the methods further include formulating said fractions or purified compound for oral or topical administration.

In some embodiments, the cell is a cultured cell, a peripheral blood mononuclear cell (PBMC), a fibroblast, or a cell in a Caenorhabditis elegans, e.g., an ASI cell.

Also provided herein are methods of increasing phase II detoxification enzyme (P2D) gene and antioxidant enzyme gene enhancing activity in a cell, by administering to the cell an effective amount of a plant extract, e.g., a willow extract, or an active fraction thereof.

Further, the invention provides methods of increasing phase II detoxification enzyme (P2D) gene and antioxidant enzyme gene enhancing activity in a cell, by administering to the cell an effective amount of a composition comprising a plant extract, e.g., a willow extract, or an active fraction thereof.

In some embodiments, the extract or active fraction reduces or prevents oxidative damage to the cell.

In some embodiments, the cell is in a living mammal, and the extract decreases oxidative damage in a tissue of the mammal In some embodiments, the cell is a skin cell, and the extract reduces oxidative damage to the skin of the mammal.

In some embodiments, the cell is a skin cell, and the extract decreases pigmentation in the skin of the mammal resulting from exposure to ultraviolet radiation.

In some embodiments, the plant extract is applied to the skin of the mammal prior to exposure to ultraviolet radiation.

In some embodiments, the methods further include formulating extracts or purified compounds identified by a method described herein for oral or topical administration. The compounds and formulated compounds are also included.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing GFP expression induced by Green Tea extract.

FIG. 2 is a graph showing GFP expression induced by Willow extract.

FIG. 3 is a graph showing GFP expression induced by sulforaphane.

FIG. 4 is a reproduction of a thin layer chromatograph showing the separation of each of the nine fractions produced as described in Example 4, with a table describing the physical characteristics and activity of the fractions.

FIG. 5 is a set of nine photographs showing the results of a fractionation experiment as described in Example 4.

FIGS. 6 and 7 are bar graphs showing the effects of different concentrations (10, 50, or 100 μg/ml) fractionated willow extracts on Nrf2 downstream gene expression. RT-PCR with SYBR™ Green was used to detect expression of glutamate-cysteine ligase modifier subunit (GCLM, FIG. 6) and glutamate-cysteine ligase catalytic subunit (GCLC, FIG. 7).

FIG. 8 is a line graph showing the effect of willow extract supplementation on SOD1 expression.

FIG. 9 is a line graph showing the effect of willow extract supplementation on Nrf2 expression.

FIG. 10 is a line graph showing the effect of willow extract supplementation on GCLM expression.

FIG. 11 is a line graph showing the effect of willow extract supplementation on catalase expression.

FIG. 12 is a line graph showing the effect of willow extract supplementation on serum 8-hydroxy-2′-deoxyguanosine (8-OHdG) transition.

FIG. 13 is a line graph showing the effect of willow extract supplementation on serum GSH transition.

FIG. 14 is a line graph showing the effect of willow extract supplementation on serum SOD transition.

FIG. 15 is a line graph showing the effect of willow extract supplementation on Forkhead Box O1 (FOXO1) expression.

FIG. 16 is a reproduction of a thin layer chromatograph showing the separation of each of the five fractions produced as described in Example 4 and pooled as described in Example 6, with a table describing the physical characteristics and activity of the fractions.

FIG. 17 is a bar graph showing induction of the Phase II response (GCS-1::GFP expression) by fractionated willow extract. The indicated numbers of animals were exposed to the different fractions of Willow preparation. Incubations were carried out using 10 mg/ml of each material, with the exception of Fraction A (5 μg/ml). M9 was used as the control for all samples except those containing Fraction A, for which DMSO was the control. Error bars correspond to the standard deviation among multiple individual experiments.

FIG. 18 is a line graph showing protection of N2 worms from oxidative stress by willow extract (10 mg/ml), green tea extract (2 μg/ml) or willow fraction A (5 μg/mL). A representative experiment is shown, with error bars indicating the standard deviation. for 48 hours on plates (see text).

FIG. 19 is a bar graph showing induction of the Phase II response (GCS-1::GFP Expression) by Carrot and Broccoli Powders. The indicated numbers of animals were exposed to either the Carrot or Broccoli preparations, except for the indicated Control sample to the right. A representative experiment is shown.

FIGS. 20A-B are bar graphs showing the effect of willow extract on two genes whose expression is regulated by Nrf1, HO-1 (20A) and NQO1 (20B).

FIG. 21 is a bar graph showing the effect of 10 ug/ml and 100 ug/ml of willow extract on expression of the NRF2 gene in human PBMC.

FIG. 22 is a bar graph showing the effect of 10 ug/ml and 100 ug/ml of willow extract on levels of NRF2 protein in human PBMC.

FIG. 23 is a bar graph showing the effect of 1 willow extract on expression antioxidant stress levels.

FIG. 24 is a line graph showing the effect of oral administration of willow extract on TBARS in human subjects.

FIG. 25 is a bar graph showing the effect of orally administered willow extract versus placebo on antioxidant response in human skin, measured by Mean Gray Value of skin exposed to UV.

FIG. 26 is a bar graph showing the effect of topically administered willow extract versus placebo on antioxidant response in human skin, measured by Mean Gray Value of skin exposed to UV.

### DETAILED DESCRIPTION

Described herein are methods and compositions that can be used to enhance Nrf2 activity, and thus activate the Phase II detoxification system, decrease levels of 8-hydroxy-2′-deoxyguanosine (8-OHdG, a standard marker of oxidatively damaged DNA), and/or increase levels of forkhead box O1 (FOXO1) gene expression, as well as methods for identifying additional compounds present in willow and tea that also enhance Nrf2, decrease levels of 8-OHdG, and increase levels of FOXO1 gene expression.

Nrf2

Nrf2, a transcription factor, is a key factor in the oxidative stress response in mammals. Nrf2 is repressed by Keap1, GSK-3, and other mechanisms; this repression is removed in the presence of oxidative stress, at which point Nrf2 is imported into the nucleus from the cytoplasm, where it binds to an antioxidant response region of a phase II detoxification enzyme (P2D) gene. Binding of Nrf2 activates transcription of the P2D gene, thereby inducing expression of the P2D enzyme. Thus, when nuclear importation and binding of NRF2 to the gene of Nrf2 are promoted, the production of the P2D enzyme is enhanced, and antioxidant power in vivo is fortified. Nrf2-gene-knockout mice tend to be extremely affected by drug toxins and cancers, and do not respond to antioxidants used in chemical defense approaches (Chan and Kan, 1999, Proc. Natl. Acad. Sci., 96, 12731-12736; Chan et al., 2001, Proc. Natl. Acad. Sci., 98, 4611-4616; Fahey et al., 2002, Proc. Natl. Acad. Sci., 99, 7610-7615; Ramos-Gomez et al., 2001, Proc. Natl. Acad. Sci., 98, 3410-3415).

Caenorhabditis elegans, a type of nematode, has an analogous oxidative stress response system to that of mammals. This system is termed the MAPK cascade. SKN-1, a target of the MAPK cascade, is a transcription factor. Like Nrf2, GSK-3 repression of SKN-1 is relieved in the presence of oxidative stress. SKN-1 is then transported into the nucleus from the cytoplasm (e.g., in the digestive system (intestine)), binds to an antioxidant response region of a P2D gene, and activates the transcription of the P2D gene, thereby inducing expression of the P2D enzyme. Thus, SKN-1 of the nematode regulates the production of the P2D enzyme by a very similar mechanism to that of Nrf2 in mammals. Given this, a substance that promotes the nuclear importation of SKN-1, and the binding of the phase II detoxification enzyme gene to the antioxidant response region, thereby enhancing the production of the phase II detoxification enzyme in C. elegans, can be expected to enhance the production of the phase II detoxification enzyme by Nrf2 in mammals. Further, suppression of cancer and various degenerative diseases can also be expected.

To verify the expression of the P2D gene by SKN-1, a known method uses a gene in which the gcs-1 gene encoding a gamma glutamylcystein synthesis enzyme, a known P2D gene in C. elegans, and a binding target of SKN-1, can be fused with a gene encoding a reporter, e.g., green fluorescent protein (GFP) (GCS-1::GFP) (An and Blackwell, 2003, Genes & Dev., 17, 1882-1893; An et al., 2005, Proc. Natl. Acad. Sci. U.S.A., 102, 16275-16280; Inoue et al., 2005, Genes Dev., 19, 2278-2283). In this method, the fused GCS-1::GFP gene is first transferred to a nematode for transformation. Under normal conditions with low oxidative stress, the expression of the fused gene in the pharynx and ASI of C. elegans can be confirmed by fluorescence emission from GFP. Under oxidative stress conditions, this fused gene is expressed in the intestine of C. elegans. As described herein, SKN-1 activation substances, e.g., willow extract and tea extract, strongly cause the expression of the GCS-1::GFP fusion gene.

FOXO1

FOXO proteins are a family of transcription factors that are inhibited by insulin-related signaling, and are involved in many biological processes including stem cell maintenance, adipose differentiation, insulin sensitivity, defense against Reactive Oxygen species (ROS) by increasing anti-oxidant enzyme gene enhancing activity, apoptosis, tumor suppression, and longevity. Many of their well-known target genes are stress response genes, including SODs. See, e.g., Antebi, PLOS genetics 3, 1565-1571 (2007); Tothova and Gilliland, Cell Stem Cell 1, 140-152 (2007); and Accili and Arden, Cell 117, 421-476 (2004).

Willow Extracts

The willow used in the methods described herein is a plant in the genus Salix or Populus of the family Salicaceae. Examples of plants in the genus Populus include “Urajirohako yanagi” (synonyms, “Hakuyo”, “Gindoro”; P. alba), Canadian poplar (P. x Canadensis), cottonwood (P. deltoides) (synonym, “Hiroha hakoyanagi”), “Kotokake yanagi” (P. euphratica), “Oobayamanarashi” (P. tomentosa), “Chirimendoro” (P. koreana), “Doronoki” (P. maximowiczii), “Yoroppa kuroyamanarashi” (P. nigra), “Seiyo hakoyanagi” (synonym, “Italia yamanarashi”; P. nigra var. italica), “Yamanarashi” (synonym, “Hakoyanagi”, “Popura”; P. sieboldii), Balsam Poplar (P. tacamahaca), “Shina yamanarashi”, “Chosen yamanarashi”, (P. davidiana), American Poplar (P. tremuloides), and P. euramericana. Examples of plants in the genus Salix include White Willow (S. alba), “Saikoku kitsune yanagi” (S. alopochroa), “Yusuraba yanagi” (S. aurita), “Shidare yanagi” (synonym, “Ito yanagi,” S. babylonica), “Yamaneko yanagi” (synonym, “Bakko yanagi,” S. bakko), “Akame yanagi” (synonym, “Maruba yanagi,” S. chaenomeloides), “Koganeshidare” (S. chrysochoma), S. daphnoides, “Salikkusu elaeagunosu” (S. elaeagnos ‘Scopoli’), “Pokkiri yanagi” (S. fragilis), “Ookitsune yanagi” (synonym, “Kinme yanagi,” S. futura), “Kawayanagi” (synonym, “Nagaba kawa yanagi,” S. gilgiana), “Neko yanagi” (S. gracilistyla), “Koro yanagi” (S. gracilistyla var. melanostachys), “Sause” (S. humboldtiana), “Inukori yanagi” (S. integra), “Shiba yanagi” (S. japonica), “Shiro yanagi” (S. jessoensis), “Kinu yanagi” (S. kinuyanagi), “Kori yanagi” (S. koriyanagi), “Ezo yanagi” (S. rorida), “Furisode yanagi” (S. leucopithecia), “Unryu yanagi” (S. matsudana f. tortuosa), “Takaneiwa yanagi” (synonym, “Rengeiwa yanagi”), “Ooshidare yanagi” (S. ohsidare), “Ezomame yanagi” (S. nummularia ssp. Pauciflora), “Ezonokinu yanagi” (S. pet-susu), S. purpurea, “Kouhiryu”, “Miyama yanagi” (synonym, “Mineyanagi,” S. reinii), “Komaiwa yanagi” (S. rupifraga), “Onoe yanagi” (synonym, “Karafuto yanagi,” S. sachalinensis), “Kogome yanagi” (S. serissaefolia), “Shirai yanagi” (S. shiraii), Salix sp, “Tachi yanagi” (S. subfragilis), “Noyanagi” (synonyms, “Himeyanagi”), “Seiyotachi yanagi”, “Kitsune yanagi” (synonym, “Iwayanagi,” S. vulpine), and “Ezonotakane yanagi” (S. yezoalpina). Buds, leaves, fruit, branches, trunk, bark, and/or roots of the willow can be used singly or in any combination thereof, and processed as necessary to a suitable form for intake. Preferable willow is white willow, with Salix daphnoides, Salix sp, Salix purpurea, Salix fragilis, and Salix alba being particularly preferred. In some embodiments, the willow is S. alba, S. daphnoides, S. purpurea or S. fragilis.

The willow extract of the present invention is preferably extracted after the above willow is subjected to suitable treatments for extraction, as necessary, e.g., chopping, drying, and/or crushing. The treated willow as mentioned above is typically extracted, using an extractant, e.g., by standing, shaking, irradiating ultrasound, heating, and/or applying pressure, independently or in any combination thereof, as necessary. In some embodiments, the preferred procedure is to immerse the willow in an extractant, followed by shaking or stirring. In some embodiments, the willow extract is fractionated, as described herein, and the fractions with the highest activity are used in the compositions described herein.

Aqueous and organic solvents are typically used as extractants, and can be used singly or in combination thereof. Examples of organic solvents include ethanol, propanol, isopropanol, butanol, and like lower alcohols, polyethylene glycol, propylene glycol, 1,3-butylene glycol, dipropylene glycol, and like polyhydric alcohols; ethyl acetate, butyl acetate, and similar esters; acetone, methyl ethyl ketone, and like ketones; and CO₂ and similar supercritical fluids. In some embodiments, the preferred extractants include water, ethanol and mixture thereof. In some embodiments, water is used as the extractant.

The temperature at which these manipulations are performed can also be altered. The extraction temperature is usually from 3° C. to the boiling point of the extractant used. The extraction time varies, depending, e.g., on the kind of extractant, extraction temperature, and/or the form of willow, but is typically from an hour to 7 days, and preferably from 2 hours to 3 days. Pressure can be applied, if required. In some embodiments, the willow extract is prepared using boiling water.

The extract can be used without modification in the compositions and methods described herein. The extract can also be used as dissolved in, e.g., water or organic solvents, e.g., after being concentrated, desiccated, exsiccated, and/or freeze-dried; after being subjected to purification treatments such as decolorization, deodorization, and/or desalting, insofar as the effects of the extract are not impaired; and/or after being subjected to fraction treatments, e.g., liquid-liquid distribution chromatography, and column chromatography. Alternatively, the willow extract can be contained in a suitable carrier, e.g., liposomes or microcapsules.

In some embodiments, the Retention factor (Rf) of the fraction is determined, and a fraction with an Rf value higher than 0.5, e.g., higher than 0.6, 0.7, 0.75, or 0.78, is selected. In some embodiments, the fractions useful in the present methods do not contain significant amounts of salicin.

Tea Extracts

The tea used in the methods and compositions described herein can include, e.g., green tea, Oolong tea, black tea, or Pu-erh tea (all of which are derived from Camellia sinensis). Any part of the plant, e.g., flowers, leaves, and/or branches can be used, either singly or in any combination thereof, and processed as necessary to a suitable form for intake. In some embodiments, the leaves are used alone. In some embodiments, the preferred tea is green tea.

The tea extracts described herein are generally prepared after the tea is subjected to suitable treatments for extraction as necessary, e.g., chopping, drying, and/or crushing. The treated tea is then typically brought into contact with an extractant, and extracted, e.g., by standing, shaking, irradiating ultrasound, heating, and/or applying pressure, independently or in any combination thereof. In some embodiments, the tea is immersed in an extractant, followed by shaking or stirring.

Aqueous and organic solvents are typically used as extractants, either singly or in any combination thereof. Examples of organic solvents include, but are not limited to, ethanol, propanol, isopropanol, butanol, and like lower alcohols, polyethylene glycol, propylene glycol, 1,3-butylene glycol, dipropylene glycol, and like polyhydric alcohols; and CO₂ and other supercritical fluids. They can be used singly or in combination thereof. Preferable extractants are water and ethanol. In some embodiments, the extractant is about 65 to 85% aqueous ethanol, e.g., about 70-80% ethanol in water.

The extraction temperature is usually from 3° C. to the boiling point of the extractant used. The extraction time varies, depending on, e.g., the kind of extractant, the extraction temperature, and/or the form of tea, but is typically from an hour to 7 days, e.g., from 2 hours to 3 days. Pressure can further be applied, if required. Furthermore, an antioxidant substance such as ascorbic acid can be added to the extractant beforehand, as necessary, for a stable extraction of active components.

In some embodiments, the tea extract is fractionated, as described herein, and the fractions with the highest activity are used in the compositions described herein.

The extract can be used without modification in the compositions and methods described herein. The extract can also be used dissolved in water, organic solvents, etc. after being concentrated, desiccated, exsiccated, or freeze-dried; after being subjected to purification treatments, e.g., decolorization, deodorization, or desalting, insofar as the effects of the extract are not impaired; and/or after being subjected to fraction treatments such as liquid-liquid distribution chromatography, and column chromatography. Alternatively, the tea extract can be used as contained in a suitable carrier, e.g., microcapsules or liposomes.

Broccoli Powder

The broccoli used in the methods described herein is a plant of the Cabbage family, Brassicaceae (formerly Cruciferae) in the genus Brassica oleracea. Flowers, buds, stems, and leaves of the broccoli can be used singly or in any combination thereof, and processed as necessary to a suitable form for internal or external use. Preferably, both flower buds and stems are used. Broccoli powder is preferably extracted after the above broccoli is subjected to suitable treatments to prepare for extraction, as necessary, e.g., chopping, drying, and/or crushing. The treated broccoli as mentioned above is then typically squeezed and/or extracted, using an extractant, e.g., water, ethanol, or a mixture thereof, by standing, shaking, irradiating ultrasound, heating, and/or applying pressure, independently or in any combination thereof, as necessary. In some embodiments, the preferred procedure is to squeeze a puree of a large mass of flower heads of broccoli. In some embodiments, the broccoli extract is fractionated, as described herein, and the fractions with the highest activity are used in the compositions described herein.

In some embodiments, the extract can be used without modification in the compositions and methods described herein. The extract can also be used as dissolved in, e.g., water or organic solvents, e.g., after being concentrated, desiccated, exsiccated, and/or freeze-dried; after being subjected to purification treatments such as decolorization, deodorization, and/or desalting, insofar as the effects of the extract are not impaired; and/or after being subjected to fraction treatments, e.g., liquid-liquid distribution chromatography and/or column chromatography.

Carrot Powder

The carrot used in the methods described herein is a plant in the genus Daucus carota. Roots, leaves, and stems of the carrot can be used singly or in any combination thereof, and processed as necessary to a suitable form for internal or external use. Preferable a root is used. Carrot powder is preferably extracted after the above carrot is subjected to suitable treatments to prepare for extraction, as necessary, e.g., chopping, drying, and/or crushing. The treated carrot as mentioned above is then typically squeezed and/or extracted, using an extractant, e.g., water, ethanol or mixtures thereof, e.g., by standing, shaking, irradiating ultrasound, heating, and/or applying pressure, independently or in any combination thereof, as necessary. In some embodiments, the preferred procedure is to squeeze a puree of a root of carrot. In some embodiments, the carrot extract is fractionated, as described herein, and the fractions with the highest activity are used in the compositions described herein.

The extract can be used without modification in the compositions and methods described herein. The extract can also be used as dissolved in, e.g., water or organic solvents, e.g., after being concentrated, desiccated, exsiccated, and/or freeze-dried; after being subjected to purification treatments such as decolorization, deodorization, and/or desalting, insofar as the effects of the extract are not impaired; and/or after being subjected to fraction treatments, e.g., liquid-liquid distribution chromatography, and column chromatography.

Compositions

The compositions described herein can include one or more plant extracts, e.g., carrot, broccoli, willow, and/or tea extract, and/or active fractions or agents derived therefrom, typically at about 0.0001 to 95% by weight, preferably 0.001 to 70% by weight, and more preferably 0.01 to 30% by weight. In some embodiments, a useful composition comprises some or all of the more active fractions, e.g., fractions 1+2 or fractions 1+2+3, of the willow extract prepared as described in Example 4, below. Further, the compositions described herein can contain additives usable in the fields of, e.g., cosmetics, medicine, or food, so long as the activity of the compound is not significantly adversely affected.

Pharmaceutical Compositions for Oral Administration

In one aspect, the present invention includes pharmaceutical compositions including the extracts and active fractions as described herein. In addition to one or more plant extracts and/or active fractions or agents derived therefrom, the compositions described herein can further contain orally acceptable carriers, additives, etc. The compositions can be used in various forms, e.g., forms suitable for oral intake, e.g., liquid preparations; tablets, granules, fine granules, powders, and like solid preparations; capsules containing said liquids or solid preparations; oral sprays; and troches. These form preparations can be produced by standard methods. The preparations are preferably in the forms of pills (particularly tablets), capsules, parvules, powders, or granules, more preferably pills or capsules. Orally-acceptable additives and carriers used in the pharmaceutical preparation field can also be included in the compositions. Examples are given as below, but not limited thereto. Excipients include, e.g., sugar alcohols (e.g., maltitol, xylitol, sorbitol, or erythritol), lactose, white sugar, sodium chloride, glucose, starch, carbonates (e.g., calcium carbonate), kaolin, crystalline cellulose, silicic acid, methylcellulose, glycerol, sodium arginate, gum arabic, talc, phosphates (e.g., calcium secondary phosphate, calcium dihydrogen phosphate, sodium hydrogen phosphate, dibasic potassium phosphate, potassium dihydrogen phosphate, calcium dihydrogen phosphate, or sodium dihydrogen phosphate), calcium sulfate, calcium lactate, or cacao butter. Viscosity controlling agents include, e.g., simple syrup, glucose liquid, starch liquid, and gelatin solution. Binders include, e.g., polyvinyl alcohol, polyvinyl ether, polyvinyl pyrrolidone, cross polyvinylpyrrolidone, hydroxypropylcellulose, low-substituted hydroxypropylcellulose, hydroxypropyl methylcellulose, hydroxyethyl cellulose, carboxyvinyl polymer, crystalline cellulose, powdered cellulose, crystalline cellulose-carmellose sodium, carboxymethylcellulose, shellac, methylcellulose, ethylcellulose, potassium phosphate, powdered gum arabic, pullulan, pectin, dextrin, corn starch, alpha-starch, hydroxypropyl starch, gelatin, xanthan gum, carragheenan, tragacanth, powdered tragacanth, and macrogoal. Disintegrators include, e.g., dry starch, sodium arginate, agar powder, laminaran powder, sodium hydrogencarbonate, calcium carbonate, polyoxyethylene sorbitan fatty acid esters, sodium lauryl sulfate, stearin acid monoglyceride, starch, and lactose. Disintegration inhibitors include, e.g., white sugar, stearin acid, cacao butter, hydrogenated oil, etc.; absorption enhancers such as quarternary ammonium salt, and sodium lauryl sulfate. Adsorbents include, e.g., starch, lactose, kaolin, bentonite, and colloidal silicic acid. Lubricants include, e.g., refining talcs, stearate, boric acid powder, and polyethylene glycol. Emulsifiers include, e.g., sucrose fatty acid ester, sorbitan fatty acid ester, enzymatically treated lecithin, zymolysis lecithin, and saponin. Antioxidants include, e.g., ascorbic acid and tocopherols. Acidulants include, e.g., lactic acids, citric acids, gluconic acids, and glutamic acids. Fortifiers include, e.g., vitamins, amino acids, lactates, citrates, and gluconates. Plasticizers include, e.g., silicon dioxide. Sweeteners include, e.g., sucralose, acesulphame potassium, aspartame, and glycyrrhizin. Perfumes include, e.g., peppermint oil, eucalyptus oil, cinnamon oil, fennel oil, clove oil, orange oil, lemon oil, rose oil, fruit flavor, mint flavor, peppermint powder, dl-menthol, and 1-menthol. Oligosaccharides include, e.g., lactulose, raffinose, and lactosucrose. Preparation solvents include, e.g., sodium acetate.

Further, solid preparations such as tablets can be coated with typical coatings as necessary to prepare, e.g., sugar-coated tablets, gelatin film-coated tablets, enteric-coated tablets, film-coated tablets, double layer tablets, or multi-layer tablets. Liquid preparations may be in the form of water-based or oil-based suspensions, solutions, syrups, or elixirs, and can be prepared by standard methods, e.g., using typical carriers and/or additives as known in the art and/or described herein.

Nutraceutical Compositions for Oral Administration

Also included in the present invention are nutraceutical compositions comprising one or more plant extracts and/or active fractions or agents derived therefrom combined with, e.g., edible carriers, food ingredients, or food additives. Such compositions are prepared by methods known in the art. Examples of such nutraceuticals include liquid foods such as beverages, and solid foods such as bars, cakes, tablets, granules, chewable tablets. Alternatively, they can be semisolid, e.g., yogurt or yogurt-like consistency. Specific examples of such food forms include, without limitation, liquid beverages such as juices, soft drinks, and teas; powdered beverages such as powdered juices or powdered soups; snacks such as chocolates, candies, chewing gums, ice creams, jellies, cookies, biscuits, corn flakes, chewable tablets, film sheets, wafers, gummies, rice crackers, and buns with bean-paste filling; seasonings such as dressings, sauces, etc.; breads, pastas, konjakmannans, fish pastes (e.g., kamaboko), seasoned sprinkles, oral sprays, and troches.

The nutraceuticals can also include various additives and carriers known in the art. For example, live microorganism such as lactic acid bacteria, inactivated microorganisms, other probiotics, vitamins, botanical medicines, other plants such as herbs, and extracts thereof, can be used as additives. Examples of carriers include sugar alcohols, excipients, binders, emulsifiers, antioxidants, acidifiers, fortifiers, anti-caking agents, lubricants, sweeteners and flavorings.

The nutraceutical compositions can be used, e.g., as health foods, functional foods, designated health foods, nutrition functional foods, or foods for the treatment of a condition in a subject, e.g., a disease or symptoms of aging.

Oral Care Products

The plant extracts and active fractions thereof described herein can be used in compositions for oral care such as tooth pastes, tooth powders, liquid dentifrice, gel dentifrice, prophylaxis paste, mouth sprays, and mouth wash. Methods for preparing such compositions, and suitable carriers and additives, are known in the art.

Compositions for Topical Administration

In addition to one or more plant extracts, and/or active fractions or agents derived therefrom, the compositions described herein can further contain externally acceptable carriers, additives, etc. The compositions can be used in various suitable for application to the skin, e.g., aqueous solutions, solubilized topical compositions, powder dispersions, water oil 2 layer compositions, water oil powder 3 layer compositions, oil/water emulsions, water/oil emulsions, water/oil/water emulsions, gels, aerosols, mists, capsules, tablets, granules and powders. These form preparations can be produced by standard methods. The preparations are preferably in the forms of aqueous solutions, oil/water emulsions, water/oil emulsions, water/oil/water emulsions, gels, aerosols, or mists. Externally-acceptable additives and carriers used in the pharmaceutical or cosmetic preparation field can also be included in the compositions. Examples are given as below, but not limited thereto. Excipients include, e.g., anionic surfactants (e.g., alkyl sulfate, polyoxyethylene alkyl ether sulfate, alkyl alaninate, alkyl glutamate, alkyl isethionate, alkyl sarcosinate or soap), cationic surfactants, amphoteric surfactants (e.g., alkyl betaine, amidopropyl betaine, or imidazolinium betaine), nonionic surfactants (e.g., polyoxyethylene hydrogenated castor oil, sorbitan fatty acid esters, polyoxyethylene sorbitan fatty acid esters, polyoxyethylene alkyl ester, block copolymer, fatty acid ester, alkyl glyceryl ether, lecithin, glycerin fatty acid ester, polyglycerine fatty acid ester, saponin, sugar ester, or alkanolamide), oily substances (e.g., mineral oil, squalane, lanolin, petrolatum, plant oil, animal oil, ceresin, fatty acid ester, or higher alcohol), polyhydric alcohols (e.g., propylene glycol, 1,3-butylenes glycol, glycerin, 1,2-hexanediol, pentylene glycol, or polyoxyethylene glycol), polymers (e.g., polysiloxane, carboxyvinyl polymer, polyvinyl ether, polyvinyl pyrrolidone, hydroxypropyl cellulose, hydroxypropyl methyl cellulose, hydroxyethyl cellulose, carboxymethyl cellulose, polyethylene glycol, pullulan, pectin, dextrin, or xanthan gum), powders (e.g., kaolin, crystalline cellulose, talc, or bentonite), organic acids, and inorganic acids. Other various ingredients (e.g., cyclosiloxane, polyvinyl alcohol, proteins, hydrolyzed protein, peptides, amino acids, ultraviolet absorbents, antiseptics, pH adjusters, wetting agents, vitamins, medicinally-effective ingredients, preservatives, colorant, or perfume) that are suitable for incorporation into cosmetics, quasi-drugs, drugs and the like may be incorporated so far as no significant detrimental influence is thereby imposed on the objects of the present invention, e.g., there is not a significant reduction in the activity of the active ingredient. Qasi-drugs have a mild effect on the body, but are neither intended for the diagnosis, prevention or treatment of disease, nor to affect the structure or function of the body.

Products can also be of any type conventionally used for external application to skin, including, for example, facial cosmetics such as lotions, milky emulsions, creams and packs; cosmetics such as foundations, blushers, lipsticks, eye shadows, eye liners and sunscreens; body cosmetics, e.g., lotions and creams; skin cleansing cosmetics such as make-up removers, face cleansers and body shampoos; bath preparations; and hair care preparations such as shampoos and conditioners.

Effective Doses

An effective dose of the compositions described herein can be determined using methods known in the art, e.g., based on in vitro studies and animal experiments. In some embodiments, the dose of a plant extract to be administered internally (e.g., as an oral composition) will be about 50 to 2000 mg, e.g., about 100 to 1000 mg, per day per adult. Further, the oral composition can be taken in one to several portions a day, before meals (e.g., within 5 minutes), between meals, after meals (e.g., within 5 minutes), or with meals. In some embodiments, the oral doses are taken with meals or after meals. In some embodiments, the dose of a plant extract to be administered externally (e.g., in a topical preparation such as a cream or lotion) will be about 0.0001 to 95% by weight of the preparation, preferably 0.001 to 70% by weight, and more preferably 0.001 to 30% by weight. In some embodiments, the topical preparations can be applied one or more times per day.

Uses

The compositions described herein, or discovered by a method described herein, are useful in the treatment of subjects who are in need of enhancement of Phase II detoxification activity. For example, it is believed that oxidative stress may play an important role in the etiology of degenerative diseases, which are generally characterized by progressive morphological changes and progressive loss in normal metabolic activity in the cells of the tissue. In some embodiments, the degenerative disease may be characterized by, e.g., aberrant levels of glutathione, or any Phase II enzyme present in the diseased cells or tissue. These abnormal levels may be either causal or symptomatic of the degenerative disease. The phrase “degenerative disease,” as used herein, refers to physiological conditions characterized by the death of normal cells in the affected tissue, not due to tumor growth or acute toxic insult. Examples of degenerative disorders include, but are not limited to, diabetes, chronic liver failure, chronic kidney failure, Wilson's disease, congestive heart failure, atherosclerosis, and neurodegenerative diseases, e.g., Parkinson's Disease, Alzheimer's Disease, Huntington's Disease, amyotrophic lateral sclerosis, multiple sclerosis, epilepsy, myasthenia gravis, neuropathy, ataxia, dementia, chronic axonal neuropathy and stroke. The treatments described herein can be used to treat subjects with a pre-existing degenerative condition, or to prevent or delay the onset or development of disease in subjects who are pre-disposed to a degenerative disorder.

In addition, the compounds are useful in the treatment of an effect of aging in a subject, e.g., on the skin of the subject. Thus, the compositions described herein can be used to treat, e.g., wrinkles, unwanted pigmentation, rough and dry skin, or dull skin.

Methods of Screening

The discovery that compounds present in tea and willow extracts are enhancers of Nrf2/Skn-1 activation of P2D genes provides the basis for methods for identifying the active compound in those extracts. A number of assays can be used in these methods, e.g., native or engineered Skn-1 activity in C. elegans, and reporter gene constructs in any suitable cell, e.g., a mammalian cell expressing Nrf2. A genomic screen for activators of the antioxidant response element is described in Liu et al., Proc. Natl. Acad. Sci. U.S.A., 104(12):5205-5210 (2007). The reporter constructs include an antioxidant response element linked to typical minimal promoter sequences along with any detectable reporter element, e.g., a fluorescent protein such as green fluorescent protein (GFP) or a variant thereof, e.g., red fluorescent protein (RFP), blue fluorescent protein (BFP), yellow fluorescent protein (YFP) or enhanced GFP (eGFP); luciferase, chloramphenicol acetyltransferase (CAT), or beta-galactosidase. Methods for designing, selecting, and making such constructs are well known in the art, see, e.g., Sambrook et al., Molecular Cloning: A Laboratory Manual, New York, Cold Spring Harbor Laboratory Press (1989).

Antioxidant Response Element (ARE)

AREs are cis-acting regulatory enhancer elements (core consensus sequence: 5′-GTGACnnnGC-3′) found in the 5′ flanking region of many phase II detoxification enzymes. AREs are activated by reactive oxygen species, as well as other electrophilic agents, and by binding of Nrf2. Genes regulated by AREs include the P2Ds heme oxygenase-1, glutathione synthesis enzymes, glutathione S-transferases, and NAD(P)H:quinone oxidoreductase 1 (NQO1), glutamylcysteine synthesis enzymes(e.g., glutamate-cysteine ligase modifier subunit (GCLM), glutamate-cysteine ligase catalytic subunit (GCLC)), and catalase, and the antioxidant enzyme superoxide dismutase (e.g., SOD1).

Phase II Detoxification Enzymes

There are six types of Phase II conjugation reactions, including glucuronidation, sulfation, methylation, acetylation, amino acid conjugation and glutathione conjugation. The reaction catalyzed by the enzyme rhodanese (the transfer of a sulfur ion to cyanide to form thiocyanate) will also be considered a Phase II reaction herein. See U.S. Pat. No. 6,812,248, incorporated herein by reference in its entirety. In some embodiments, the screening methods described herein include detecting one or more of these conjugation reactions in a cell, and quantifying such activity, to determine whether a fraction includes a compound that increases the conjugation reaction.

### Fractionated Samples

In general, the methods described herein include the use of fractions of the extracts, e.g., a subset of all of the components present in the extract. Such fractions can be produced using any method known in the art, and can be prepared based on any one or more physical properties of the components of the extract, e.g., size, pH, pI, solubility, or charge. A number of methods for fractionating the extracts described herein are known in the art, e.g., protein and peptide fractionation techniques, including but not limited to immunodepletion (affinity removal), gel electrophoresis, reverse phase chromatography, gel or other filtration, ion exchange, column chromatography, e.g., using silica gel, isoelectric focusing, e.g., immobilized pH gradient isoelectric focusing (IPG IEF), and solution-phase, pI-based fractionation systems fractionate proteins or peptides by pI. Liquid-liquid fractionation or solid-liquid fractionation methods can also be used.

See, e.g., Jin et al., Biotechnol J. 2006 February; 1(2):209-13; Si et al., Bioassay-guided purification and identification of antimicrobial components in Chinese green tea extract. J Chromatogr A. 2006; 1125(2):204-10; Paveto et al., Anti-Trypanosoma cruzi activity of green tea (Camellia sinensis) catechins. Antimicrob Agents Chemother. 2004; 48(1):69-74; Kinjo et al., Activity-guided fractionation of green tea extract with antiproliferative activity against human stomach cancer cells. Biol Pharm Bull. 2002; 25(9):1238-40; Satoh et al., Black tea extract, thearubigin fraction, counteracts the effect of tetanus toxin in mice. Exp Biol Med (Maywood). 2001; 226(6):577-80; Sagesake-Mitane et al., Platelet aggregation inhibitors in hot water extract of green tea. Chem Pharm Bull (Tokyo). 1990; 38(3):790-3; Jassbi, Z Naturforsch [α]. 2003; 58(7-8):573-9. Secondary metabolites as stimulants and antifeedants of Salix integra for the leaf beetle Plagiodera versicolora; and Wildermuth and Fall, Biochemical characterization of stromal and thylakoid-bound isoforms of isoprene synthase in willow leaves. Plant Physiol. 1998; 116(3):1111-23, inter alia.

### EXAMPLES

The invention is further described in the following examples, which do not limit the scope of the invention described in the claims.

### Example 1

### Preparation of Extracts

A number of extracts were prepared for use in the present experiments, including green tea, white willow, pine bark, and broccoli (sulforaphane) extracts.

Green Tea Extract

Green Tea extract (Thaea Sinensis, Emil Flachsmann AG/Frutarom, Haifa, Israel, Prod. No. 85.942) was prepared by immersing green tea leaves in a solution wherein 0.0025% ascorbic acid was dissolved in ethanol (80%), and stirring slowly for four hours at room temperature, followed by filtration of the extract to remove the tea leaves. The extractant was then removed using a decompressed concentrator, thereby preparing a green/brown extract to which dextrin was added as an excipient, and the mixture was powdered for use in the experiment.

A 2 mg/mL DMSO solution containing Green Tea extract was prepared. Since DMSO can cause oxidative stress in C. elegans at higher concentrations, the Green Tea extract was added to M9 saline medium (42 mM Na₂HPO₄; 22 mM KH₂PO₄; 86 mM NaCl; 1 mM MgSO₄*7 H₂O) so as to have a final concentration of 2 μg/mL for use as a test material. The final concentration of DMSO was 0.1%. Such a low concentration of DMSO did not cause oxidative stress in C. elegans.

White Willow Extract

White Willow extract (Salicis Cortex, Emil Flachsmann AG/Frutarom, Haifa, Israel, Prod. No. 0085816) was prepared by immersing dried commercial White Willow bark and sprouts in purified water, and stirring slowly for four hours at room temperature, followed by filtration of the extract to remove solid substances. The extractant was then removed using a decompressed concentrator, thereby preparing a brown extract to which gum Arabic was added as an excipient, and the mixture was powdered for use in the experiment.

Willow extract dissolved in M9 saline medium at a concentration of 10 mg/mL was used as a test material.

Pine Bark Extract

Pine Bark extract was prepared by extracting from dried pine bark in hot water for 3 to 4 hours for use in the experiment.

Pine bark extract was dissolved in DMSO so as to have a concentration of 2 mg/mL, and was added to M9 saline medium to have a final concentration of 2 μg/mL for use as a test material.

Sulforaphane

Further, sulforaphane (an active derivative of broccoli sprouts) was used as a positive control. Sulforaphane was dissolved in acetonitrile, and then diluted to 1 mg/mL with M9 saline medium for use as a test material.

### Example 2

### Preparation and Expression of gcs-1::GFP Fusion Constructs

It is well known that the enzyme encoded by gcs-1 gene (CGS-1), a phase II detoxification enzyme (P2D), is a rate-limiting enzyme for glutathione synthesis in vivo, and the gcs-1 gene is a target gene of SKN-1 that regulates the detoxification and antioxidation of the second generation.

A nucleic acid encoding the GCS-1 promoter (as described in An and Blackwell, 2003, Genes & Dev., 17, 1882-1893) was fused with a sequence encoding green fluorescent protein (GFP) to prepare a reporter construct (gcs-1::GFP) using standard molecular biology techniques. This fusion construct gene was transferred into C. elegans (An and Blackwell, 2003, Genes & Dev., 17, 1882-1893; Mello, et al., EMBO J. 1991. 10(12):3959-70), and the obtained transformed C. elegans were used in the experiment. Under normal, less oxidative stress conditions, the gcs-1::GFP construct is expressed in the pharynx area and ASI of C. elegans, where fluorescence emission of GFP can be measured.

As a comparison, a mutant of an SKN-1 binding site in the gcs-1 gene promoter (gcs-1Δ2Mut3) (see An and Blackwell, 2003, Genes & Dev., 17, 1882-1893) was fused with the sequence encoding GFP to prepare a gene (gcs-1Δ2Mut3::GFP gene), which was tested in the same manner as with gcs-1::GFP gene. Since this mutant gene cannot bind with SKN-1, it was unable to express GCS-1 regulated by SKN-1. The SKN-1 regulation of GCS-1 expression is hence verified when GFP was expressed in the above fused gene, but was not expressed in the mutant gene.

### Example 3

### Willow and Tea Extracts Enhance GCS-1::GFP Expression

Experiments were conducted to study the influence of the various extracts prepared in Example 1 on expression of the GCS-1::GFP reporter construct (described in Example 2) in C. elegans, following the method according to An and Blackwell, 2003, Genes & Dev., 17, 1882-1893.

Before each experiment, approximately 20 L4 stage worms carrying the GCS-1::GFP reporter construct transgene were picked to NGM plates (a type of agar media, see Brenner, Genetics, 1974 May; 77(1):71-94), containing OP50 bacteria (an E. coli strain, food for C. elegans), and were allowed to grow for 2 to 3 days. The worms were transferred together with a saline medium (M9) for treatment with the test materials to a microcentrifuge tube by flooding the NGM plate surface with the saline medium (M9). The microcentrifuge tube was then centrifuged, and a supernatant was removed. The same procedure was repeated again to wash the worms. This washing removed the bacteria which had been given as food.

Once the worms were washed, they were added for incubation in the specified test materials at the specified concentrations for the following amounts of time. Initially, incubation time was 30 minutes, and was later extended to include 60, 90, and 120 minutes. After treatment for said given times, the worms were washed in M9 at least twice, transferred back to an NGM plate containing bacteria, and allowed to recover for about 30 minutes. The worms were then mounted on slides, and scored for GFP expression levels under a microscope. GFP expression levels in the intestines of each worm were evaluated based on three scores; high, medium, and low expressions. A high score was given for worms with GFP expression through out the intestine. GFP expression midway up the intestine was scored medium. A low score was given to worms with very little or no GFP expression in their intestine.

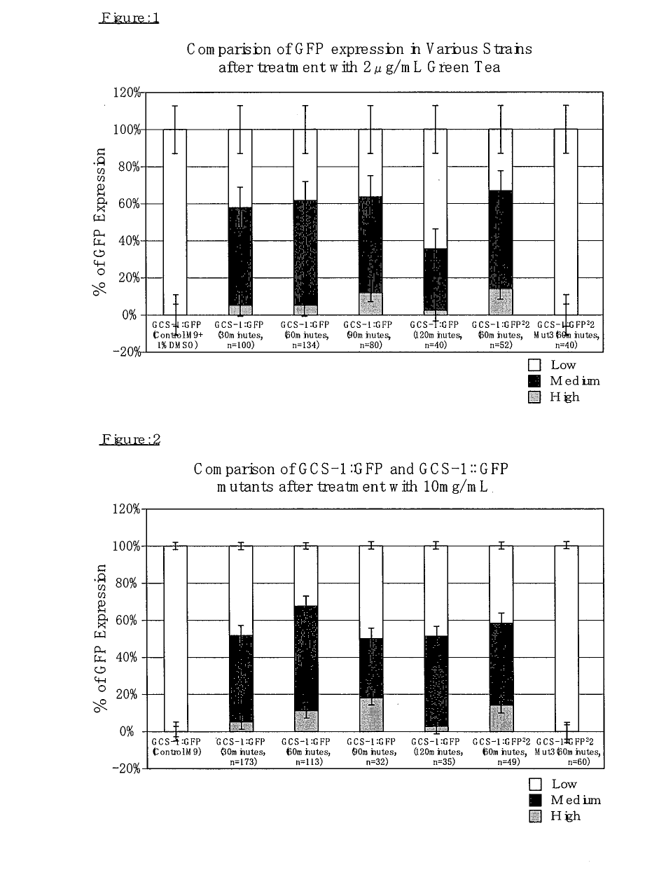

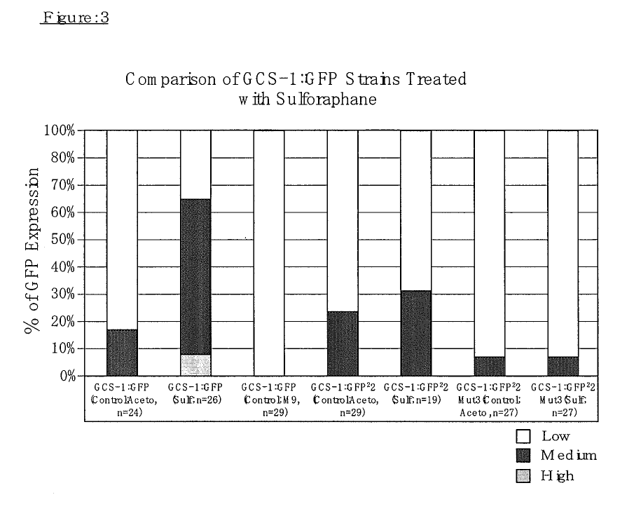

The results, shown in FIGS. 1 and 2, demonstrate that treatment with either the green tea extract or willow extract dramatically increased GFP expression driven by the gcs-1 promoter, as compared to control conditions.

The willow extract significantly increased the number of worms given a medium or high score in expression. In these experiments, the negative control M9 saline solutions did not induce GFP expression in the intestine, with all worms being scored as low. The maximum response was seen with 60-minutes treatment. However, no effect was seen with sulforaphane after 30, 60 or 90 minutes of the incubations. For this reason, treatment with sulforaphane was given a longer incubation time, and the effect was first seen at after 6 hours. Data shown in FIG. 3 revealed that the tea extract and willow extract significantly induced GCS-1::GFP expression in an obviously shorter time compared to sulforaphane.

Worms in which the gcs-1Δ2::GFP gene was transferred (GCS-1Δ2::GFP worms) were used to test whether the effects of the test materials depend on SKN-1. This promoter mutant transgene lacks pharyngeal gcs-1 gene expression; however, it maintains SKN-1-dependent expression in the ASI neurons and intestine. With both green tea extract and willow extract, the GCS-1Δ2::GFP worms displayed the same expression level as the GCS-1::GFP worms under the condition of 60-minutes incubation. The mutant transgene CGS-1Δ2mut3::GFP worms were also used to determine whether the response was SKN-1 dependent. This gene, a variant of gcs-1Δ2::GFP gene (gcs-1Δ2mut3::GFP gene), lacks the SKN-1 binding site in its promoter region, because of which GFP is not expressed in the pharynx, the ASI neurons, or the intestine, under normal and stress conditions. When these mutants (CGS-1Δ2mut3::GFP worms) were treated with either the green tea or willow extract, GFP expression was not observed. Comparisons of the results with test materials in CGS-1::GFP worms, CGS-1Δ2::GFP worms, and CGS-1Δ2mut3::GFP worms revealed that, with the green tea extract and willow extract, GCS-1::GFP expression was seen in the ASI neurons and intestine, substantially no GCS-1::GFP expression was seen in the pharynx, thus GCS-1::GFP expression was regulated by SKN-1 binding. With sulforaphane, however, the comparison showed that about half of GCS-1::GFP expression was seen in the pharynx, and GCS-1::GFP expression was not partially regulated by SKN-1.

### Example 4

### Fractionation of Willow Extracts

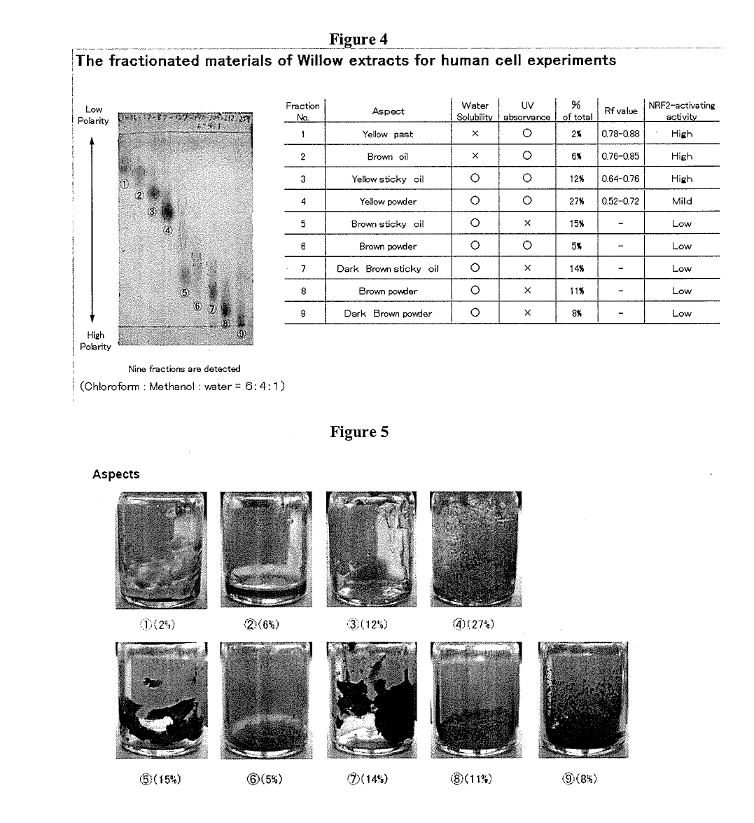

In order to identify a fraction having the most effect on Phase II activation from the original willow extract, a column chromatography method was used. For example, Silica gel packed column can be used for the fractionation of the willow extract. FIGS. 4 and 5 show representative results of a fractionation experiment performed using column chromatography.

In order to prepare the separation column, 400 g of silica gel 60 (70-230 mesh ASTM, obtained from Merck) was suspended in methanol and poured into the column. After that, the methanol was replaced by a chloroform:methanol (10:1) solution. Five grams of original willow extract were re-suspended in water and loaded on the upper side of the silica gel surface. As the first elution solvent, about 1.5 L chloroform:methanol (10:1) was used for elution of the materials. The eluted solution was collected into the fraction tube for each 20 mL. The liquid phase was a chloroform:methanol (10:1) solution, followed by Upper layer of chloroform:methanol:water (7:3:1), then chloroform:methanol:water (6:4:1), Chloroform:methanol:water (5:5:1), and finally a methanol wash was used. Then, the materials were assayed by thin layer chromatography (TLC) methods. The eluted solutions were developed on the TLC plate (TLC plate Silica gel 60 F254 provided by Merck) using a solution of chloroform:methanol:water (6:4:1). In order to detect the fractions, 50% sulfuric acid was splayed on the TLC plate, which was then heated at 250 degrees.

More effective materials were further defined by the retention factor (Rf) value of TLC development. Rf, is defined as the distance traveled by the compound divided by the distance traveled by the solvent. The Rf values for fractions 1-4 is shown in Table 1. The Rf value of the effective fraction was located from 0.5 to 0.9. The Rf value of the most effective fractions was from 0.6 to 0.9.

TABLE 1
Rf values of fractions 1-4

| Fraction | Rf value |
|---|---|
| 1 | 0.78-0.88 |
| 2 | 0.76-0.85 |
| 3 | 0.64-0.76 |
| 3 | 0.52-0.72 |

Fractions having an Rf value from 0.6 to 0.9 can also be isolated by the other methods. For example, the reversed phase particle (C2, C8, C18: C means carbon) can also be used instead of silica gel. In this case, the more effective fractions can be eluted using a water:methanol or water:ethanol solution, and the identity of the fractions determined by their Rf value.

Gel chromatography methods, which separate species by molecular weight, and ion absorbance gel chromatography methods, which separate by the polarity of the molecules, can also be used for fractionation. Liquid-liquid fractionation or solid-liquid fractionation methods can also be used instead of column chromatography.

Before column chromatography is performed, activated charcoal, for example an activated charcoal column, can be used as pretreatment, to remove color (e.g., to remove chlorophyll from the dark strange color extracts).

FIGS. 4 and 5 show the results of one fractionation experiment, using column chromatography. The Column was a solid phase Silica gel 60 (70-230 mesh ASTM, from Merck). The liquid phase was a chloroform:methanol (10:1) solution, followed by Chloroform:methanol:water (7:3:1), then chloroform:methanol:water (6:4:1) Chloroform:methanol:water (5:5:1) and a final methanol wash. The extracts shown in FIG. 5 were prepared as follows: extracts 1 to 3 were extracted using chloroform:methanol (10:1) solution, extract 4 was extracted using Chloroform:methanol:water (7:3:1), extracts 5 to 7 were extracted using chloroform:methanol:water (6:4:1), extract 8 was extracted using Chloroform:methanol:water (5:5:1), and then extract 9 was extracted using methanol. The solvent was then removed using a standard evaporator. The “aspect” refers to the appearance of the fraction by visual inspection. 0.05 g of material was put into 5 ml water and voltexed. Water solubility was measured if it was clearly soluble in room temperature; in FIG. 4, an open circle indicates that the material was clearly soluble, while an “X” indicates not clearly soluble (e.g., particulate matter was present). UV spots were observed using a UV detector, and UV absorbance was measured by visual inspections. Also in FIG. 4, an open circle indicates that a UV spot was observed, while an “X” indicates that no spot was observed. Percentages shown in FIG. 4 are by weight. “High” gene expression was assigned if the gene expression of both GCLM and GCLC were significantly high compared with control, and the relative value was more than 4 (at 100 μg/ml); “Mild” was assigned if gene expression of both GCLM and GCLC were significantly high compared with control, and the relative value was less than 4 (at 100 μg/ml); and “Low” meant that gene expression of both GCLM and GCLC were not significantly high compared with control, and the relative value was less than 4 (at 100 μg/ml).

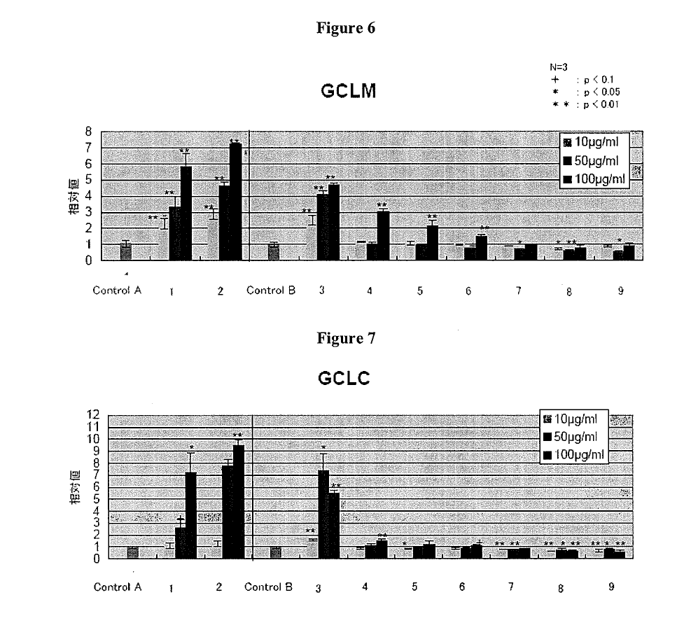

FIGS. 6 and 7 show the results of evaluation of the effects of fractionated willow extracts on Nrf2 downstream gene expression. Human fibroblast cells were contacted with the nine fractionated willow extracts shown in FIGS. 4 and 5, at concentrations of 10 μg/ml, 50 μg/ml, or 100 μg/ml, and incubated for 24 hours. RT-PCR with SYBR™ Green was used to detect expression of glutamate-cysteine ligase modifier subunit (GCLM, FIG. 6) and glutamate-cysteine ligase catalytic subunit (GCLC, FIG. 7). PPIA was also evaluated as an internal control gene. The results demonstrated that fractions 1, 2, and 3 contained the highest amount of NRF2-activating activity.

### Example 5

### Administration of Willow Extracts to Human Subjects

This example describes a small trial conducted to examine the anti-oxidative ability of willow extracts in healthy human volunteers aged about 26-45 years, with an average age of 34.2 years. 16 subjects were enrolled (7 males and 9 females), 3 dropped out during the trial.

The subjects were administered willow extract from Ask Intercity Co., Ltd. This willow extract was prepared by immersing dried commercial willow bark and sprouts in purified water with heating, whereof the willow bark and young branches are “White willow bark” based on European Pharmacopeia and Commission E Monograph. The doe regimen was 6 capsules/day (for a total of 800 mg of willow extract per day) for a period of two weeks (followed by a wash out period of two weeks). Each subject then underwent a clinical examination, including:

(i) measurement of anti-oxidant associated gene expression in peripheral blood mononuclear cells (PBMCs) isolate from heparinized blood using a Ficcoll-Conray gradient—Nrf2, GCLM, forkhead box O1 (FOXO1), SOD1, and catalase. GAPDH was used as an internal control (measured using RT-PCR at 0, 1, 2, and 4 weeks);

(ii) serum anti-oxidative index—levels of 8-hydroxy-2′-deoxyguanosine (8-OHdG), GSH, SOD, 8-isoprostane, and TRAP (measured at 0, 1, 2, and 4 weeks);

(iii) blood biochemistry—total protein, GOT, GPT, total cholesterol, HDL-C, LDL-C, triglyceride, ALP, albumin, A/G ratio, γ-GTP, amylase, urea nitrogen, uric acid, creatinine, and atherosclerosis index (measured at 0, 2, and 4 weeks);

(iv) blood hemocyte count—Blood glucose, HbA1c, WBC, RBC, Hb, Ht, platelet, basophil, acidphol, neutrophil, leukocyte, monocyte, MCV, MCH, and MCHC (measured at 0, 2, and 4 weeks); and

(v) others—Insulin, adiponectin, IGF-1 and salicylic acid (measured at 0, 2, and 4 weeks).

The subjects' profiles are shown in Table 2.

TABLE 2
Subject Profiles

| Subject | | | | | |
|---|---|---|---|---|---|
| No. | Gender | Age | Intake rate | Data defect | Note |
| 1 | F | 27 | 100.0 | — | |
| 2 | F | 45 | 91.7 | — | |
| 3 | F | 35 | 95.2 | — | |
| 4 | F | 26 | 107.1 | 2 w | |
| 5 | F | 40 | 84.5 | 1 w | Antibiotics during |
| | | | | | supplementation |
| 6 | F | 26 | 100 | — | |
| 7 | F | 35 | 101.2 | — | |
| 8 | F | 38 | 100 | | Contraceptive |
| | | | | | during |
| | | | | | supplementation |
| 9 | F | 28 | 100 | — | |
| 10 | M | 36 | 98.8 | — | |
| 11 | M | 28 | 100 | — | |
| 12 | M | 43 | 102.4 | — | |
| 13 | M | 31 | 103.6 | — | |
| 14 | M | 39 | 81.0 | — | |
| 15 | M | 26 | 92.9 | — | |
| 16 | M | 44 | 100.0 | — | |

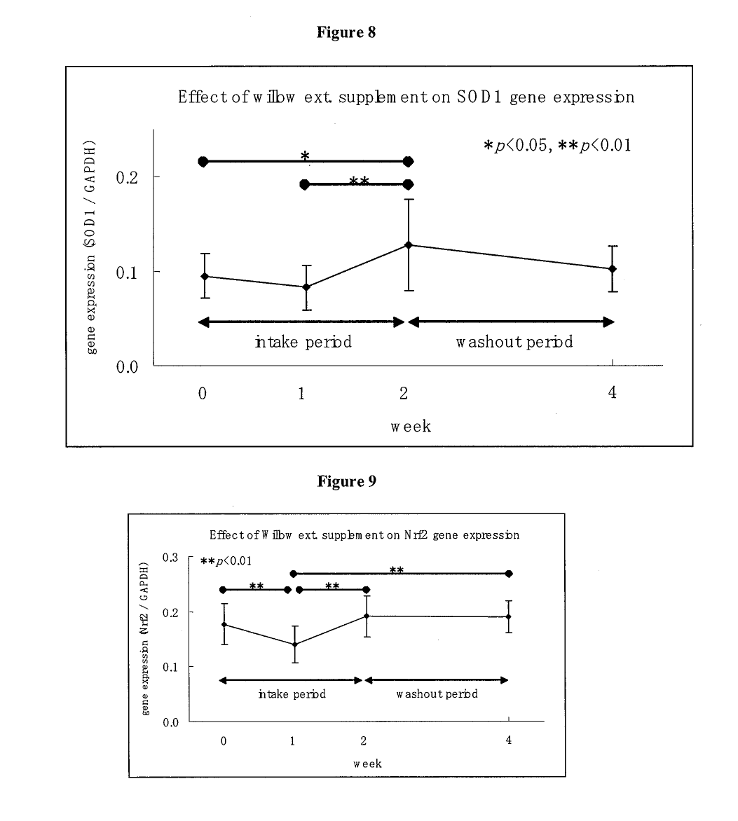

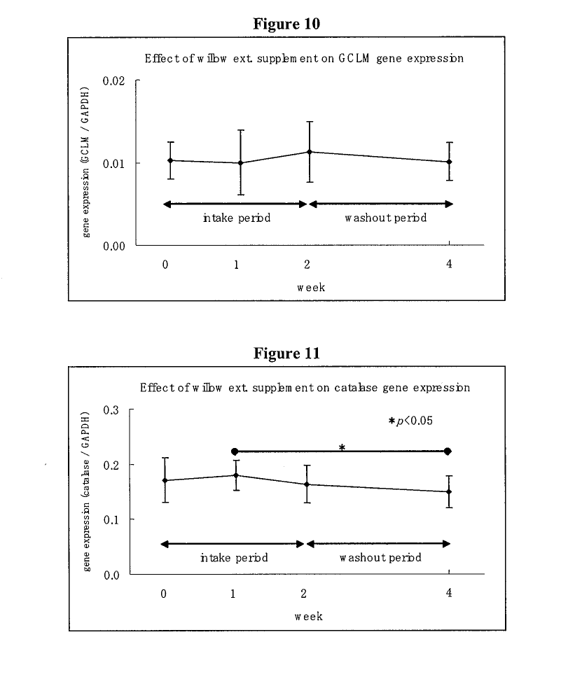

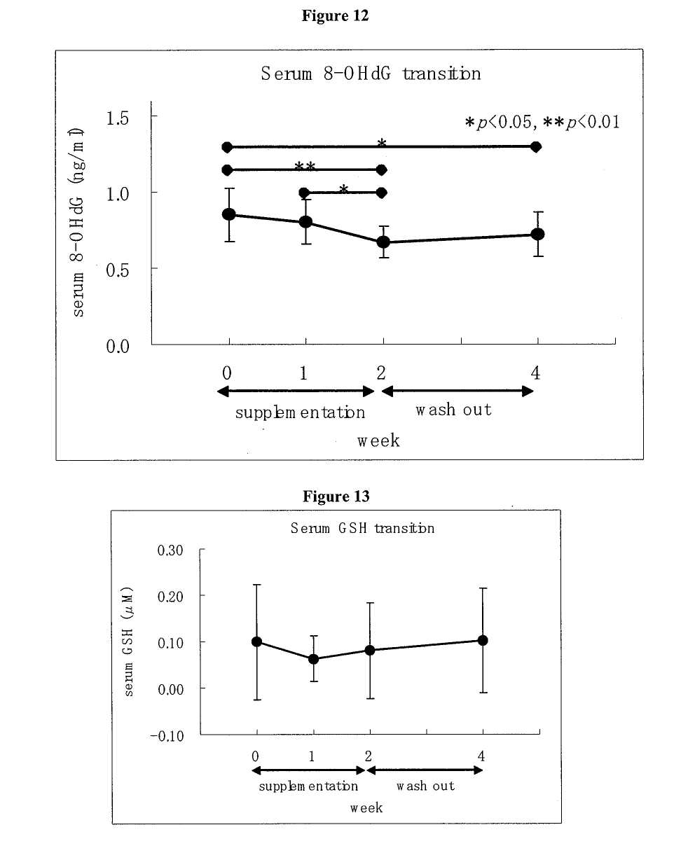

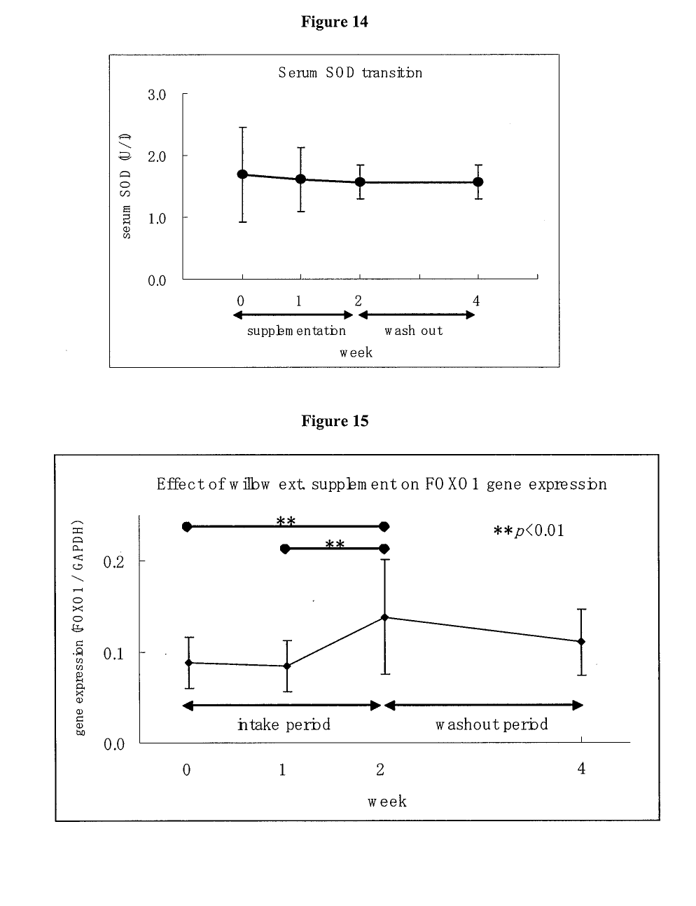

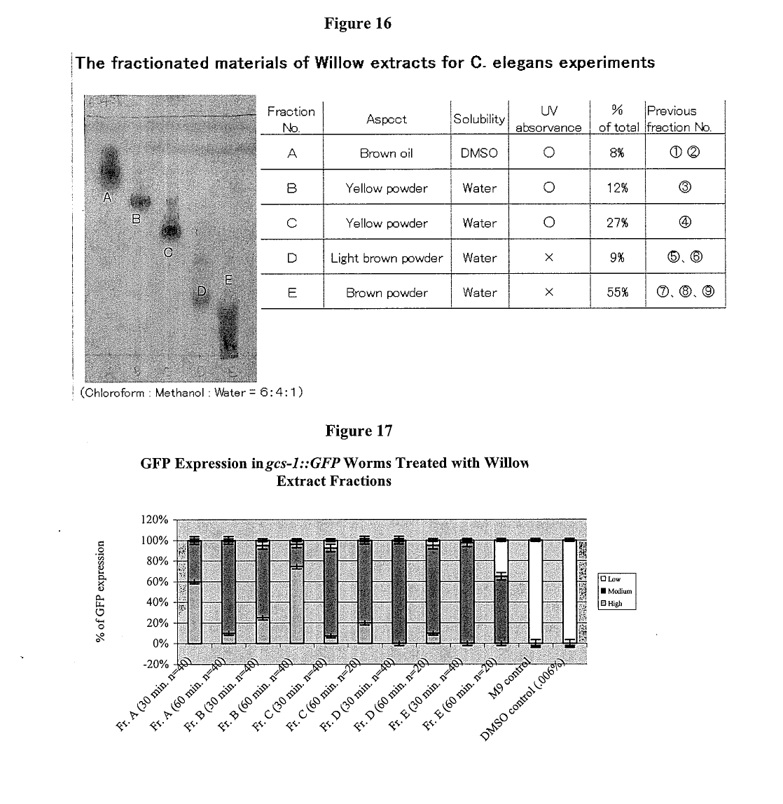

The results are shown in FIGS. 8-15. First, as shown in FIG. 8 and Table 2, two weeks of willow extract supplementation induced SOD1 gene expression in PBMC. As shown in FIGS. 9-11, gene expression of Nrf2 in PBMC significantly declined one week after intake, while neither of the downstream genes GCLM or catalase changed significantly during the supplementation period (catalase gene expression slightly increased during the first week of the intake period, but returned to baseline during the second week of the intake period, and catalase gene expression decreased during residual periods). As shown in FIG. 12, supplementation with willow extract significantly reduced serum 8-OHdG levels in two weeks and the effect persisted after the treatment was ended. However, as shown in FIGS. 13 and 14, GSH and SOD transition levels in serum did not change throughout the test period.

In contrast, as shown in FIG. 15, FOXO1 mRNA was significantly increased in PBMC after two weeks of supplementation with the willow extract. FOXO1 is a transcription factor known to regulate detoxification and antioxidant gene expression, including SOD1.

### Example 6

### Hydrophobic Willow Extracts Increase GCS-1 Expression in Vivo

This example describes experiments performed to investigate whether certain fractions of willow extract induce the SKN-1/Phase 2 detoxification pathway in living C. elegans. Fractionated products of willow extract were prepared as described above, and pooled as shown in Table 3 to form fractions A-E (Fr. A-Fr. E).

TABLE 3
Fractions Combined

| New Fraction Name | Previous Fraction |
|---|---|
| A | 1, 2 |
| B | 3 |
| C | 4 |
| D | 5, 6 |
| E | 7, 8, 9 |

As above, these experiments were carried out in C. elegans using a gcs-1 transgene that had been fused with green fluorescent protein (GFP) (GCS-1::GFP). gcs-1 encodes an enzyme that is rate-limiting for glutathione synthesis, and is a particularly well characterized and diagnostic target gene for the Phase II master regulator SKN-1 (An and Blackwell, Genes Dev. (17):1882-93 (2003); An et al., Proc. Natl. Acad. Sci. U.S.A. (102):16275-80 (2005); Inoue et al., Genes Dev. (19):2278-83 (2005); Tullet et al., Cell. 132:1025-38 (2008)). Under oxidative stress conditions, SKN-1-dependent gcs-1 expression is induced in intestinal cells.

The GCS-1::GFP worms were subjected to treatment with the various fractions and GFP expression levels in the intestines of the animals were observed. In each individual experimental trial, approximately 20 L4 stage worms were picked to fresh plates containing OP50 bacteria. After 2-3 days, the animals were treated with the materials. The worms were transferred to a microfuge tube by flooding the plate containing the worms with M9 (a saline medium), and using a pipette to transfer them to the tube. After a quick spin, the M9 was removed and the animals were washed one more time with M9. This washing removes any of the remaining bacteria. Once the worms had been washed they were incubated with the preparations provided for either 30 or 60 minutes. After the incubation, the worms were washed 2 more times in M9 and transferred to a fresh NGM plate containing bacteria and allowed to recover for about 30 minutes. The worms were then mounted on slides and scored for GFP expression under the microscope. One of three scores (high, medium or low expression) was given based on the levels of GFP expression in the intestine, as described in (An and Blackwell, Genes Dev. (17):1882-93 (2003); Tullet et al., Cell. 132:1025-38 (2008)). A high score was given for animals with GFP throughout the intestine. GFP expression midway through the intestine is an example of a medium score. A low score was given to worms which had very little or no GFP expression in their intestine.

First, induction of the gcs-1 transgene reporter after treatment with Willow extract fractions was examined (FIG. 17). Treatment with Fraction A resulted in the highest increases in intestinal GFP expression when compared to control after 30 minutes of treatment (FIG. 17). Material from Fraction A was administered at a low concentration (5 μg/mL) because it was dissolved in DMSO, which can elicit an oxidative stress response at higher concentrations. The low final concentration of DMSO in the Fraction A sample (006% DMSO) did not elicit a stress response in the gcs-1 worm (Control, FIG. 17). Fractions B-E, which were administered in M9 medium at 10 mg/ml, also induced intestinal GCS-1::GFP expression, with each successive fraction resulting in a slightly lower level of induction than the previous one (FIG. 17). Interestingly, Fraction B elicited a comparably robust response when administered at 5 μg/mL (not shown), suggesting that its potency is comparable to that of Fraction A.

In summary, all of the Willow fractions were characterized by gcs-1 induction activity, with Fractions A and B being the most potent and the others showing successively less activity.

### Example 7

### Protective Effects of Green Tea and Willow Extracts

This example describes experiments performed to investigate whether treatment with previously analyzed green tea and willow extract materials enhances survival of C. elegans under oxidative stress and normal conditions.

The following materials were tested for whether they protected the animal from exposure to oxidative stress:
Willow extract
Green Tea extract
Fractionated product of Willow Extract (Fr. A)

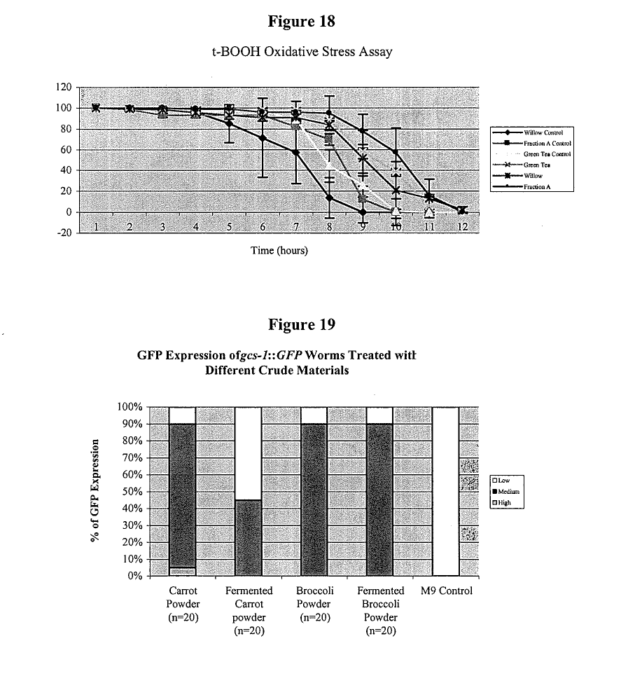

In each experiment, the worms were exposed to oxidative stress by treatment with tert-Butyl hydroperoxide solution (t-BOOH), a lipid-soluble source of peroxide radicals (FIG. 18). L4 stage worms were picked to plates containing the material to be tested, or a control. Those plates had been seeded with bacterial cultures that had been spun down and resuspended in 5 ml of the respective material. After incubation for 24 or 48 hours at 20° C., the worms were moved along with a small amount of bacteria to plates containing 15.4 mM t-BOOH. Worms were then checked for movement and pharyngeal pumping every hour until all were dead. Each analysis was performed in triplicate using approximately 20 worms per plate. The following controls were used: willow: OP50 bacterial food resuspended in LB; green tea: OP50 resuspended in LB containing 0.1% DMSO; willow fraction A: OP50 resuspended in LB containing 0.006% DMSO.

After 48 hours, all three materials provided protection against oxidative stress, as indicated as increased survival compared against the appropriate controls. The Willow extract was soluble in LB at a concentration of 10 mg/mL. The negative control for this group (OP50 bacteria alone) provided no protection against t-BOOH with all worms dead by the 9^{th} hour. In contrast, some worms treated with Willow extract lived 12 hours (FIG. 18). The Green Tea extract also provided protection, even though its final concentration being 2 ug/mL because it was soluble only in DMSO (0.01%). Finally, treatment with Willow extract Fraction A also provided significant protection.

In contrast, exposure to these same extract materials for only 24 hours did not provide any protection against t-BOOH stress.

In summary, treatment with each of the preparations tested protected C. elegans from a subsequent oxidative stress challenge (treatment with t-BOOH). Conditions are being established for an analysis of effects on lifespan.

### Example 8

### Effects of Carrot and Broccoli Extracts

Experiments as described above in Example 6 were carried out using the following materials in place of the willow or tea extracts:
Carrot Powder
Fermented carrot powder
Broccoli powder
Fermented broccoli powder

Worms were treated with 10 mg/mL of each respective preparation for 30 minutes. Treatment with carrot powder resulted in the highest increases in intestinal expression of the GFP reporter compared with the other materials after a 30-minute treatment (FIG. 19). Each of these was soluble in M9 saline at a concentration of 10 mg/mL. Again, the negative M9 control showed no effects on GFP in the intestine, with all worms being scored as low.

In conclusion, all of the tested materials induced gcs-1 expression moderately in comparison with the M9 control.

### Example 9

### Effects of Willow Extract on Expression of Genes Regulated by Nrf2 (HO-1 and NQO1)

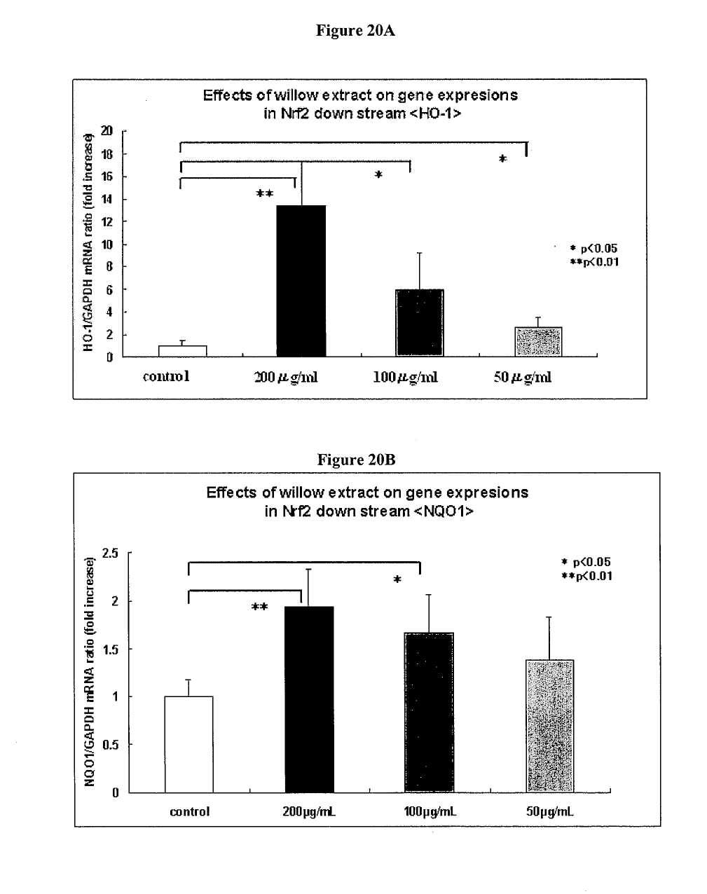

To examine the effect of willow extract on expression of genes regulated by Nrf2, HUVECs were purchased from Sanko Junyaku (Japan), and cultured at 37° C. and 5% CO₂ in MCDB131 supplemented with 10% FBS, 10 ng/mL FGF and 100 U/mL penicillin, and 100 μg/mL streptomycin in type I collagen coated plate. HUVECs at 4th passage were seeded on 12-well type I collagen coated plates. When the cells reached confluence, they were starved for the subsequent 24 hours in medium containing 2% FBS without FGF. After the starvation period, the medium was exchanged to fresh media containing willow extract (Ask Intercity Co., Ltd.) dissolved at the desired final concentration (see FIGS. 20A-B). Total RNA was extracted from the cells using a Total RNA Mini Kit (BIO-RAD, USA) at 6 hours after the introduction of the willow extract. Single-strand cDNA was synthesized from 0.5 μg of total RNA using PrimeScript RT reagent Kit (Takara, Japan). Quantitative analysis of heme oxygenase 1 (HO-1) and NADPH dehydrogenase quinone 1 (NQO1) mRNA was performed by real-time PCR using ABI 7500 Fast Real-Time PCR System (Applied Biosystems, Japan). Premix Ex Taq (Takara, Japan) and Assay-on-Demand, Gene Expression Products were used for the quantitative real-time PCR analysis. All the quantitative data were normalized by the expression level of glyceraldehyde-3-phosphate dehydrogenase (GAPDH).

The results, shown in FIGS. 20A-B indicate that willow extract increases expression of HO-1 and NQO1, genes that are regulated by Nrf-2, in a dose-dependent manner.

### Example 10

### Effect of Willow Extract on Nrf2 Expression in Isolated PBMC

Blood from a healthy volunteer was collected into a heparinized tube, and diluted by adding an equal quantity of PBS (−). Peripheral blood mononuclear cells (PBMCs) were isolated from the diluted blood using Ficcoll-Conray gradient method. 1.0×106 of PBMCs were cultured in the presence or absence of willow extract from Ask Intercity Co., Ltd, at 37° C. and 5% CO2 in RPMI1640 supplemented with 10% FBS, 100 U/mL penicillin, and 100 μg/mL streptomycin. Total RNA was extracted from the cells using a Total RNA Mini Kit (BIO-RAD, USA) 4 hours after the incubation. Single-strand cDNA was synthesized from total RNA using PrimeScript RT reagent Kit (Takara, Japan). Quantitative analysis of glutamate-cysteine ligase modifier subunit (GCLM) and NF-E2 related factor 2 (NRF2) mRNA was performed by real-time PCR using ABI 7500 Fast Real-Time PCR System (Applied Biosystems, Japan). Premix Ex Taq (Takara, Japan) and Assay-on-Demand, Gene Expression Products were used for the quantitative real-time PCR analysis. All the quantitative data were normalized by the expression level of glyceraldehyde-3-phosphate dehydrogenase (GAPDH).

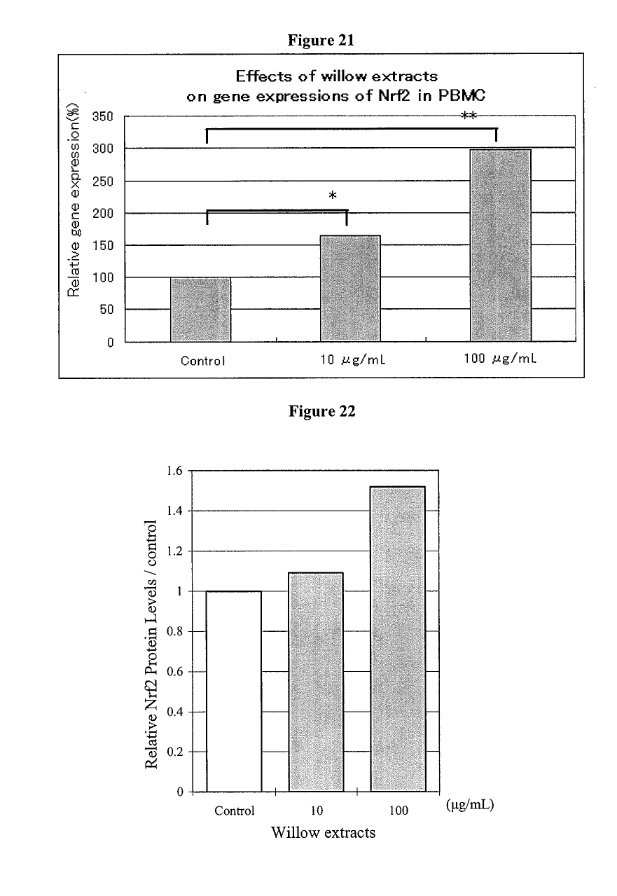

The results, shown in FIG. 21, indicate that willow extract increases expression of Nrf-2 in a dose-dependent manner in human PBMC.

### Example 11

### Evaluation of Nrf2 Translocation from Cytoplasm to Nucleus: Nrf2 Activation Effects in Skin Fibroblasts

Skin fibroblasts used for this example were abdominal fibroblasts derived from a 50-year-old white woman (hereinafter abbreviated as HDF50) (Cell Applications, Inc.). The culture medium used was MEM(+) medium prepared by adding 50 mL of standard fetal bovine serum (SIGMA) and 5.0 mL of Antibiotic Antimycotic Solution (100×) (SIGMA) to 500 mL of MEM-Eagle medium (SIGMA) and mixing.

HDF50 was cultured in MEM(+) medium at 37° C. in a 5% CO₂ incubator. When HDF50 reached a confluent state, cells were isolated to count the number of cells by a hemocytometer (Bürker-Türk hemocytometer). The cells obtained were diluted in MEM(+) medium to make 1.9×10⁵ cells/15 mL. After adding a material to be evaluated to the diluted medium, the mixture was incubated further for 24 hours at 37° C. in a 5% CO₂ incubator. Thereafter, the cells were isolated again to obtain a cell nuclear extract using a Nuclear/Cytosol Fractionation Kit. Protein in the cell nuclear extract was determined using a Protein Assay Rapid Kit and the protein concentrations were adjusted to make the quantity of protein equivalent among all the samples. A sample thus prepared was mixed with equal volume of Laemmli sample buffer containing 5% 2-mercaptoethanol and boiled. A supernatant obtained from the boiled mixture was subjected to gel electrophoresis. Immediately after the completion of electrophoresis, the gel was transferred to a nitrocellulose membrane attached to the kit using an iBlot gel transfer device and a band of Nrf2 was detected around 100 Kda using Amersham ECL Plus Western Blotting Detection System. Furthermore, after removing antibodies using a Re-Blot Western Blot Recycling Kit, laminA/C was detected as the control in a similar manner. As for the Nrf2 band, the gel image was scanned and the density of the Nrf2 band was quantitated using Scion Image Software (NIH's Windows version) to calculate the relative Nrf2 protein levels as a control.

The results, shown in FIG. 22 indicate that willow extract increases levels of Nrf-2 protein in a dose-dependent manner in human fibroblasts.

### Example 12

### Evaluation of ability to Prevent Oxidative Stress

After culturing in the medium with diluted materials for 24 hours, HDF50 were incubated for 30 minutes in Dulbecco's phosphate buffered saline (SIGMA) (hereinafter abbreviated as D-PBS) containing 5 mM H2O2. Thereafter, the medium was replaced by MEM(+) medium and the cultivation was continued for an additional 3 hours at 37° C. in a 5% CO₂ incubator.

After completion of the cultivation, the number of live cells(A) was determined using a hemocytometer (Bürker-Türk hemocytometer) and the rate of cell viability was calculated comparing with the number of live cells of no H2O2 addition condition(B) according to the following equation:

The rate of cell viability=[(A)/(B)]×100(%)

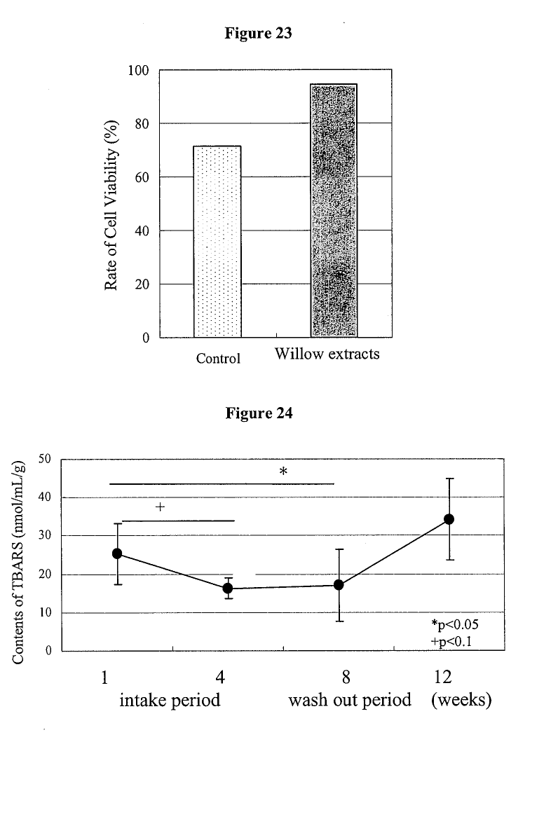

The results, shown in FIG. 23, indicate that willow extract has a positive effect on preventing oxidative stress.

### Example 13

### Antioxidation in Human Skin (Oral Intake)

To evaluate the stimulatory action of willow extract on antioxidation in human skin, willow extract (Ask Intercity Co., Ltd.) was given orally to 7 healthy males aged 32 to 43 years old at a dose of 800 mg per day. The intake period was 4 weeks and the washout period was 8 weeks. Antioxidant activity was measured by the amount of lipid peroxide in sebum. Sebum was obtained four times in total, immediately before the start of intake, after the completion of the intake period, during the washout period (at week 4) and after the completion of the washout period.

Sebum was obtained by injecting acetone/ether (1:1) solution into a cylinder with inner diameter of 4 cm placed closely on the collection site. The sebum samples obtained from three sites of the back of each subject were combined and lipid peroxide was determined using TBARS Assay Kit (OXITEK). The fluorescent measurement in the determination of lipid peroxide was performed using a RF540 spectrofluorophotometer (Shimadzu, Japan) and the amount of lipid peroxide was obtained as a MDA value (Contents of TBARS(nmol/mL/g)).

The results, shown in FIG. 24, indicate that the willow extract significantly and reversibly increased antioxidant activity after oral administration for four weeks.

To further evaluate the stimulatory action of orally administered willow extract on antioxidation in human skin, sixteen healthy males aged 24 to 47 years old were divided into the test group (11 males) and the placebo group (5 males). The test group was given orally 6 capsules per day (for a total of 800 mg per day of willow extracts (Ask Intercity Co., Ltd.) and crystalline cellulose). The placebo group was given orally 6 capsules per day (containing crystalline cellulose only). The intake period was 6 weeks. UV irradiation was performed twice, 2 weeks before the start of intake and 4 weeks after the start of intake. UV was irradiated on the back of each subject at 30 mJ/cm² using a solar simulator. The photos at the UV irradiation sites were taken 2 weeks after each UV irradiation. UV irradiation and photographing were performed in the placebo group at the same time as that in the test group. The amount of pigment (Mean Gray Value) was obtained using an image processing and analysis in Java Version 1.39 (NIH) after performing automatic color level correction of photo image data using color chart in a Photoshop Element (Adobe).

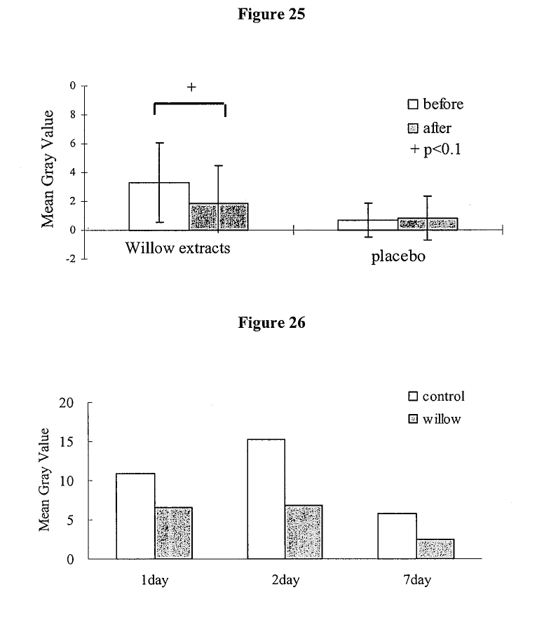

The results, shown in FIG. 25, indicate that the willow extract increased antioxidant activity after oral administration, as demonstrated by a significant reduction in the amount of pigment produced by UV radiation.

### Example 14

### Antioxidation in Human Skin (Topical Application)

This example describes the evaluation of the stimulatory action of topically administered willow extract on antioxidation in human skin. The external application period was 1 week. A test sample containing 1% of willow extract (Ask Intercity Co., Ltd.) to be tested in aqueous alcohol gel (containing 0.45% carbomer and 4.75% ethyl alcohol) was used. The placebo sample containing 0.45% carbomer and 4.75% ethyl alcohol was used. The aqueous alcohol gel was applied on the lower arm twice a day at a dose of 0.2 g/5 cm². After the completion of the application period, the application site was washed with water and dried. Then the site was irradiated with 30 to 40 mJ/cm² of UV (adjusted dependent on the UV sensitivity of panel) using solar simulator. At 6 days after UV irradiation, photos were taken at the irradiation sites. The amount of pigment (Mean Gray Value) was obtained using an image processing and analysis in Java Version 1.39 (NIH) after performing automatic color level correction of photo image data using color chart in a Photoshop Element (Adobe).

The results, shown in FIG. 26, indicate that the willow extract increased antioxidant activity after topical application, as demonstrated by a reduction in the amount of pigment produced by UV radiation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a white willow extract or an active fraction thereof, wherein the composition increases expression of one or both of a phase II detoxification enzyme (P2D) gene and an antioxidant enzyme gene in a cell.

2. The composition of claim 1, wherein the composition (i) increases expression of a P2D gene selected from the group consisting of glutamate-cysteine ligase modifier subunit (GCLM), and glutamate-cysteine ligase catalytic subunit (GCLC); (ii) increases expression of an antioxidant enzyme gene comprising superoxide dismutase 1 (SOD1); (iii) increases expression of forkhead box O1 (FOXO1): and/or (iv) decreases levels of 8-hydroxy-2′-deoxyguanosine (8-OHdG).

3. (canceled)

4. The composition of claim 1, wherein the composition is formulated for oral administration, e.g., comprises one or more orally acceptable carriers and additives.

5. (canceled)

6. The composition of claim 1, wherein the composition is formulated for topical administration, e.g., comprises one or more topically acceptable carriers and additives.

7. (canceled)

8. A method of increasing the phase II detoxification enzyme (P2D) or antioxidant enzyme enhancing activity of an extract of willow, the method comprising:
providing an extract of willow having a first level of P2D or antioxidant enzyme enhancing activity;
fractionating the extract, to obtain two or more fractions;
selecting a fraction having an Rf value of 0.5 or greater;
assaying the P2D or antioxidant enzyme enhancing activity of the fraction; and
selecting the fraction if it has a level of P2D or antioxidant enzyme enhancing activity that is higher than the first level of P2D or antioxidant enzyme enhancing activity.

9. The method of claim 8, wherein fractionating the extract comprises using one or more methods selected from the group consisting of column chromatography, liquid-liquid fractionation, and solid-liquid fractionation.

10. A method of identifying a compound that increases expression of phase II detoxification enzyme (P2D), antioxidant enzyme genes, or a forkhead box O1 (FOXO1) gene in a cell, the method comprising:
(a) providing a cell expressing (i) a P2D, antioxidant enzyme, or a FOXO1 gene or (ii) a reporter construct comprising a P2D, antioxidant enzyme, or FOXO1 gene promoter;
(b) providing a fraction of a plant extract;
(c) contacting said cell with said fraction; and
(d) detecting an effect of said fraction on expression of the P2D, antioxidant enzyme, or FOXO1 gene or reporter construct,
wherein a fraction that increases expression of the P2D, antioxidant enzyme, or FOXO1 gene or reporter construct comprises a compound that increases expression of P2D, antioxidant enzyme, or FOXO1 genes in a cell.

11. The method of claim 10, further comprising:
(e) selecting a fraction that increases expression of the P2D, antioxidant enzyme, or FOXO1 gene or reporter construct, and further dividing said fraction, to produce two or more subfractions;
(f) providing a cell expressing a P2D, antioxidant enzyme, or FOXO1 gene or a reporter construct comprising a P2D, antioxidant enzyme, or FOXO1 gene promoter;
(g) contacting said cell with said subfraction; and
(h) detecting an effect of said subfraction on expression of the P2D, antioxidant enzyme, or FOXO1 gene or reporter construct,
wherein a subfraction that increases expression of the P2D, antioxidant enzyme, or FOXO1 gene or reporter construct comprises a compound that increases expression of P2D, antioxidant enzyme, or FOXO1 genes in a cell.

12. The method of claim 11, further comprising repeating steps (e) through (h), until a purified compound is obtained.

13. The method of claim 12, further comprising formulating said purified compound for oral or topical administration.

14. (canceled)

15. The method of claim 10, wherein the cell is a cultured cell, a peripheral blood mononuclear cell (PBMC), a fibroblast, or a cell in a Caenorhabditis elegans, e.g., an ASI cell.

16. The method of claim 10, wherein the plant extract is a willow extract.

17. (canceled)

18. The method of any claim 8, wherein the P2D gene is selected from the group consisting of glutamate-cysteine ligase modifier subunit (GCLM), glutamate-cysteine ligase catalytic subunit (GCLC), and the antioxidant enzyme gene is superoxide dismutase 1 (SOD1).

19. The method of claim 8, further comprising
(e) selecting a fraction that increases expression of the P2D or antioxidant enzyme gene or reporter construct, and further dividing said fraction, to produce two or more subfractions;
(f) providing a cell expressing a FOXO1 gene or a reporter construct comprising a FOXO1 gene promoter;
(g) contacting said cell with said subfractions;
(h) detecting an effect of each of said subfractions on (i) expression of the FOXO1 gene or reporter construct, or (ii) levels of 8-hydroxy-2′-deoxyguanosine (8-OHdG) in the cell; and
selecting a subfraction that increases expression of the FOXO1 gene or reporter construct or reduces levels of 8-OHdG in the cell.

20.-27. (canceled)

28. A method of increasing phase II detoxification enzyme (P2D) or antioxidant enzyme gene enhancing activity in a skin cell of a mammal, the method comprising administering to the cell an effective amount of the composition of claim 1, comprising a willow extract or an active fraction thereof.

29. (canceled)

30. The method of claim 28, wherein the extract reduces oxidative damage to the cell and/or decreases pigmentation in the skin of the mammal resulting from exposure to ultraviolet radiation.

31. The method of claim 28, wherein the cell is in a living mammal, and the extract decreases oxidative damage to the skin of the mammal.

32. (canceled)

33. The method of claim 31, wherein the extract decreases pigmentation in the skin of the mammal resulting from exposure to ultraviolet radiation.

34. The method of claim 33, wherein the plant extract is applied to the skin of the mammal prior to exposure to ultraviolet radiation.

35. The method of claim 10, wherein the P2D gene is selected from the group consisting of glutamate-cysteine ligase modifier subunit (GCLM), glutamate-cysteine ligase catalytic subunit (GCLC), and the antioxidant enzyme gene is superoxide dismutase 1 (SOD1).

36. The method of claim 10, further comprising
(e) selecting a fraction that increases expression of the P2D or antioxidant enzyme gene or reporter construct, and further dividing said fraction, to produce two or more subfractions;
(f) providing a cell expressing a FOXO1 gene or a reporter construct comprising a FOXO1 gene promoter;
(g) contacting said cell with said subfractions;
(h) detecting an effect of each of said subfractions on (i) expression of the FOXO1 gene or reporter construct, or (ii) levels of 8-hydroxy-2′-deoxyguanosine (8-OHdG) in the cell; and
selecting a subfraction that increases expression of the FOXO1 gene or reporter construct or reduces levels of 8-OHdG in the cell.
